(19) United States
(12) Patent Application Publication
(10) Pub. No.: US 2025/0276142 A1
(43) Pub. Date: Sep. 4, 2025

(54) SLEEP DIAGNOSES AND THERAPIES

(71) Applicant: Halare, Inc., Sewickley, PA (US)

(72) Inventor: Anthony C. Warren, Wien (AT)

(73) Assignee: Halare, Inc., Sewickley, PA (US)

(21) Appl. No.: 19/060,908

(22) Filed: Feb. 24, 2025

Related U.S. Application Data
(60) Provisional application No. 63/560,239, filed on Mar. 1, 2024.

(51) Int. Cl. A61M 16/00

(52) U.S. Cl. CPC A61M 16/024; A61M 2230/42

(57) ABSTRACT

A system and method for diagnosing and treating sleep disorders using a portable smart device. The device detects and records physiological parameters of a user during awake and sleep states. A processor analyzes these parameters to determine at least one of a loop gain or an arousal index of the user's ventilatory control system. A trigger mechanism initiates a disturbance of the ventilatory control system during awake and sleep states and monitors the response of the ventilatory control system to such disturbance. The system can provide feedback to the user based on at least one of the determined loop gain or the arousal index and display these parameters to the user via a user interface.

### FIELD

The present disclosure generally relates to the field of sleep disorder diagnosis and therapy, and more specifically, to systems, methods, and devices for determining patient phenotypes and ventilatory control system stability using physiological parameters and response to disturbances.

### BACKGROUND

Sleep disorders, particularly Sleep Disordered Breathing (SDB), are a prevalent health concern affecting a large proportion of the population. SDB is characterized by repeated episodes of hypopnea (under-breathing) and apnea (not breathing) during sleep, leading to a reduction in blood oxygen saturation (SpO₂), arousal from sleep, and activation of the sympathetic nervous system. The predominant type of SDB is Obstructive Sleep Apnea (OSA), which affects approximately 85% of patients with SDB. OSA is a multifactorial disorder characterized by at least three primary phenotypes, including mechanical impairments of the upper airway, a low respiratory arousal threshold, and a high loop gain, which contributes to unstable ventilatory control during sleep.

Current therapies for alleviating OSA predominantly target mechanical impairments of the upper airway. These therapies include Positive Airway Pressure (PAP), Mandibular Advancement Splints (MAS), Neuromuscular Electrical Stimulation (NMES), and surgical procedures. However, these therapies are deficient for various reasons including being only targeted at treatment of one phenotype of OSA, namely mechanical impairment of the upper airways.

### SUMMARY

In one aspect, the present disclosure relates to a system for diagnosing and treating sleep disorders, comprising a portable smart device configured to detect and record physiological parameters of a user during awake and sleep states, a processor configured to analyze the physiological parameters to determine a loop gain and an arousal index of the user's ventilatory control system, and a trigger mechanism configured to initiate a disturbance of the ventilatory control system during awake and sleep states and monitor the response of the control system to such disturbance.

In embodiments of this aspect, the disclosure according to any one of the above example embodiments, the portable smart device is configured to detect and record physiological parameters including at least one of heart rate, heart rate variability, blood pressure, blood oxygen concentration, and changes in breathing patterns.

In embodiments of this aspect, the disclosure according to any one of the above example embodiments, the portable smart device is a smartphone.

In embodiments of this aspect, the disclosure according to any one of the above example embodiments, the trigger mechanism is configured to initiate the disturbance of the ventilatory control system by instructing the user to hold their breath.

In embodiments of this aspect, the disclosure according to any one of the above example embodiments, the trigger mechanism is configured to initiate the disturbance of the ventilatory control system by applying an external trigger including at least one of sound, light, and motion.

In embodiments of this aspect, the disclosure according to any one of the above example embodiments, the processor is further configured to analyze the physiological parameters to determine a recovery time duration and a number of recovery breaths following a breath-holding event.

In embodiments of this aspect, the disclosure according to any one of the above example embodiments, the processor is further configured to estimate the loop gain and the arousal index based on the physiological parameters and the response of the ventilatory control system to the disturbance.

In embodiments of this aspect, the disclosure according to any one of the above example embodiments, the processor is further configured to track the loop gain and the arousal index over time.

In embodiments of this aspect, the disclosure according to any one of the above example embodiments, the processor is further configured to provide feedback to the user based on the determined loop gain and arousal index.

In embodiments of this aspect, the disclosure according to any one of the above example embodiments, the system further comprises a user interface configured to display the determined loop gain and arousal index to the user.

In one aspect, the present disclosure relates to a method for diagnosing and treating sleep disorders, comprising the steps of detecting and recording physiological parameters of a user during awake and sleep states using a portable smart device, analyzing the physiological parameters to determine a loop gain and an arousal index of the user's ventilatory control system using a processor, initiating a disturbance of the ventilatory control system during awake and sleep states using a trigger mechanism, and monitoring the response of the control system to the disturbance.

In embodiments of this aspect, the disclosure according to any one of the above example embodiments, the portable smart device is a smartphone.

In embodiments of this aspect, the disclosure according to any one of the above example embodiments, the physiological parameters include at least one of heart rate, heart rate variability, blood pressure, blood oxygen concentration, and changes in breathing patterns.

In embodiments of this aspect, the disclosure according to any one of the above example embodiments, the trigger mechanism initiates a disturbance of the ventilatory control system by instructing the user to hold their breath.

In embodiments of this aspect, the disclosure according to any one of the above example embodiments, the trigger mechanism initiates a disturbance of the ventilatory control system by applying an external trigger including at least one of sound, light, and motion.

In embodiments of this aspect, the disclosure according to any one of the above example embodiments, the processor is further configured to analyze the physiological parameters to determine a recovery time duration and a number of recovery breaths following a breath-holding event.

In embodiments of this aspect, the disclosure according to any one of the above example embodiments, the processor is further configured to estimate the loop gain and the arousal index based on the physiological parameters and the response of the ventilatory control system to the disturbance.

In embodiments of this aspect, the disclosure according to any one of the above example embodiments, the processor is further configured to track the loop gain and the arousal index over time.

In embodiments of this aspect, the disclosure according to any one of the above example embodiments, the processor is further configured to provide feedback to the user based on the determined loop gain and arousal index.

In embodiments of this aspect, the disclosure according to any one of the above embodiments, the processor is further configured to determine the most appropriate therapy for the alleviation of sleep disturbed breathing.

In embodiments of this aspect, the disclosure according to any one of the above example embodiments, the method further comprises a user interface configured to display the determined loop gain and arousal index to the user.

The foregoing general description of the illustrative embodiments and the following detailed description thereof are merely example aspects of the teachings of this disclosure and are not restrictive.

### BRIEF DESCRIPTION OF FIGURES

So that the way the above-recited features of the present disclosure can be understood in detail, a more particular description of the disclosure, briefly summarized above, may be made by reference to example embodiments, some of which are illustrated in the appended drawings. It is to be noted, however, that the appended drawings illustrate only example embodiments of this disclosure and are therefore not to be considered limiting of its scope, for the disclosure may admit to other equally effective example embodiments.

FIGS. 1A-1D illustrate different breathing patterns as relative amplitude of airflow to the lungs plotted against time during breath-holding exercises, according to aspects of the present disclosure.

FIG. 2A depicts a nasal cannula for detecting breathing patterns, according to aspects of the present disclosure.

FIG. 2B shows abdominal belts for detecting breathing patterns, according to aspects of the present disclosure.

FIG. 2C presents a microphone and a smartphone for detecting breathing patterns, according to aspects of the present disclosure.

FIGS. 2D and 2E illustrate the processing of breathing sounds for analyzing breathing patterns including relative amplitude of airflow to the lungs plotted against time and relative amplitude of sound waves detected using a microphone during the same event respectively, according to aspects of the present disclosure.

FIG. 3 depicts a graph illustrating the calculation of estimated Loop Gain (LG) in a breathing pattern as relative amplitude of airflow to the lungs plotted against time, according to aspects of the present disclosure.

FIGS. 4A and 4B show the relationship between a number of recovery breaths and the time taken to recover after a breath-holding exercise, according to aspects of the present disclosure.

FIG. 5 illustrates different stages of sleep modes over a period of time, according to aspects of the present disclosure.

FIG. 6 illustrates the different sleep modes and the transition to an arousal state, according to aspects of the present disclosure.

FIG. 7 shows a sequence of trigger applications over time, according to aspects of the present disclosure.

FIG. 8 illustrates the response of a patient's blood oxygen saturation (SpO₂) during sleep to various external triggers, according to aspects of the present disclosure.

FIG. 9 depicts heart rate monitoring during sleep, according to aspects of the present disclosure.

FIGS. 10A-10C illustrate the application of external triggers and the impact on sleep stages, according to aspects of the present disclosure.

FIG. 11 shows the response of physiological parameters to a series of increasing intensity triggers during sleep, according to aspects of the present disclosure.

FIG. 12 depicts changes in airflow to the lung during time during a breathing exercise pattern designed to alleviate sleep disruption due to breathing control instabilities, according to aspects of the present disclosure.

FIG. 13 illustrates the activity flow of the system with decisions made to determine different therapy options to be implemented, according to aspects of the present disclosure.

### DETAILED DESCRIPTION

Various example embodiments of the present disclosure will now be described in detail with reference to the drawings. It should be noted that the relative arrangement of the components and steps, the numerical expressions, and the numerical values set forth in these example embodiments do not limit the scope of the present disclosure unless it is specifically stated otherwise. The following description of at least one example embodiment is merely illustrative in nature and is in no way intended to limit the disclosure, its application, or its uses. Techniques, methods, and apparatus as known by one of ordinary skill in the relevant art may not be discussed in detail but are intended to be part of the specification where appropriate. In all the examples illustrated and discussed herein, any specific values should be interpreted to be illustrative and non-limiting. Thus, other example embodiments may have different values. Notice that similar reference numerals and letters refer to similar items in the following figures, and thus once an item is defined in one figure, it is possible that it need not be further discussed for the following figures. Below, the example embodiments will be described with reference to the accompanying figures.

The present disclosure pertains to systems, methods, and apparatuses for diagnosing and treating sleep disorders. In some aspects, the disclosure provides a portable smart device designed to detect and record physiological parameters of a user during both awake and sleep states. The device may be configured to analyze these parameters to determine specific characteristics of the user's ventilatory control system, such as loop gain and arousal index. As used herein, “Loop Gain” may refer to a measure of the stability of the ventilatory control system, quantifying the magnitude of the ventilatory response to a disturbance. A higher loop gain may indicate a more unstable system that is prone to oscillations, while a lower loop gain may suggest a more stable system. The “Arousal Index” may refer to a measure of sleep fragmentation, typically expressed as the number of arousals per hour of sleep. An arousal may be defined as a brief awakening or shift to a lighter stage of sleep, often in response to a respiratory event or external stimulus. A higher arousal index may indicate more fragmented sleep and a lower threshold for arousal, while a lower arousal index may suggest more consolidated sleep and a higher threshold for arousal. These characteristics can provide beneficial insights into the nature of the user's sleep disorder and guide the selection of appropriate treatment strategies.

In some cases, the system may include a trigger mechanism that initiates a disturbance of the ventilatory control system during awake and sleep states. The trigger mechanisms for awake and sleep states are designed to initiate disturbances in the ventilatory control system to assess its stability and responsiveness. In the awake state, the trigger mechanism may involve voluntary actions by the user, such as breath-holding exercises. During sleep, the trigger mechanism utilizes external stimuli that can be applied without waking the user, allowing for assessment of the arousal threshold and ventilatory control system stability. Specific examples of trigger mechanisms for the awake state may include instructing the user to hold their breath for a specified duration, guided breathing exercises with varying patterns and depths, and voluntary hyperventilation followed by normal breathing. For the sleep state, trigger mechanisms may include auditory stimuli of increasing intensity (e.g., white noise, tones, or recorded sounds), visual stimuli such as brief flashes of light, tactile stimuli like gentle vibrations applied to the body, mild temperature changes in the sleep environment, subtle changes in the incline of the sleeping surface, and brief alterations in air pressure or flow through a connected breathing apparatus. The response of the control system to such disturbance can be monitored and analyzed to further inform the diagnosis and treatment of the sleep disorder. This approach may offer a non-invasive and cost-effective alternative to traditional sleep study methods, which often require complex devices and can be intrusive to the patient's sleep process.

In some aspects, the system may be used to estimate the loop gain of the ventilatory control system based on the length of a volitional breath hold and the number of recovery breaths following the breath hold. This information can be beneficial in determining whether the user's sleep disorder is due to instabilities in the ventilatory control system or other factors. In other aspects, the system may be used to estimate the arousal index of the ventilatory control system by applying external triggers during Deep or Rapid Eye Movement® sleep and monitoring the user's response to these triggers.

In some cases, the system may also be used to monitor the user's breathing patterns during breath training exercises. This can provide real-time feedback on the user's compliance with the exercises and track improvements in breathing patterns over time. Such information can be beneficial in tailoring the treatment to the specific requirements of the user and enhancing the effectiveness of the treatment.

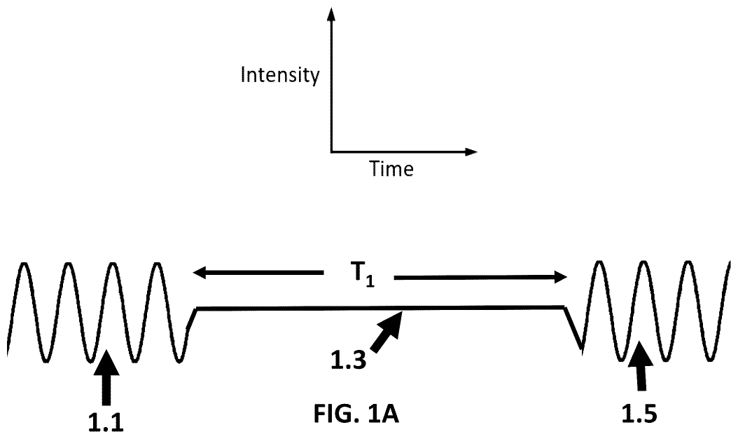

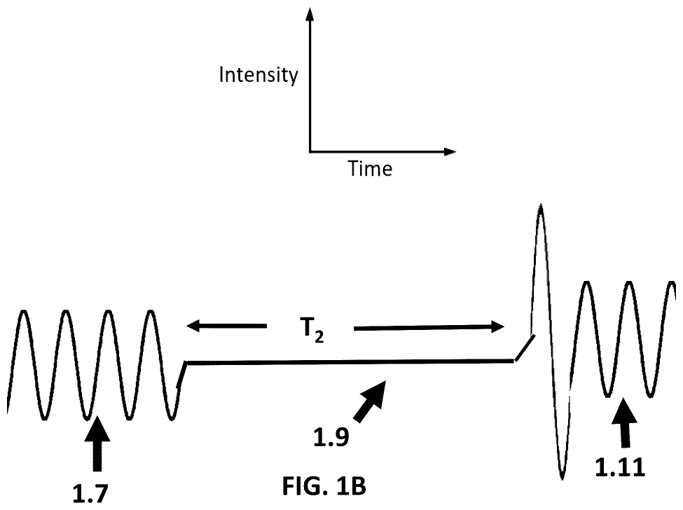

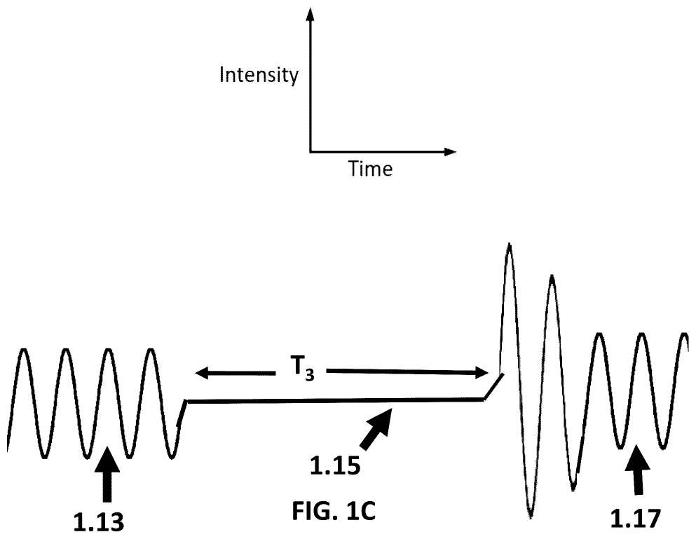

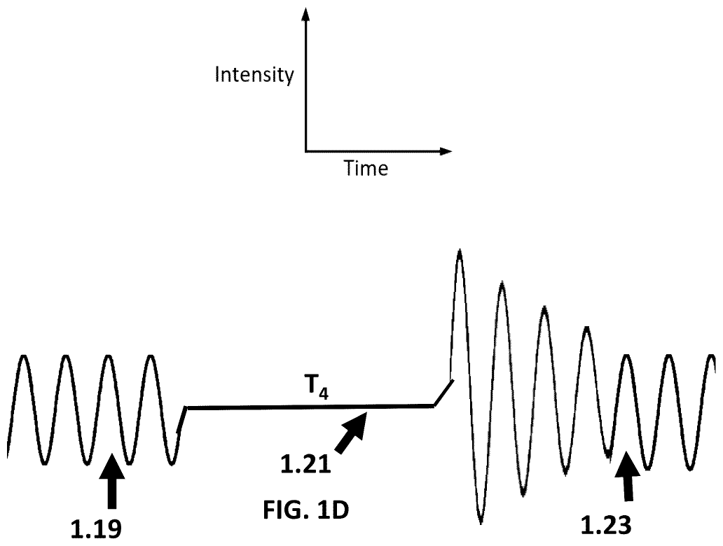

Referring to FIG. 1A, a graph is depicted representing a breathing pattern during a breath-holding exercise, over a period of time. The graph illustrates the end of a relaxed breathing sequence 1.1, followed by a breath hold duration T₁ 1.3, and then a resumption of regular breathing 1.5. The recovery time duration Tr is the period between the end of the breath hold duration 1.3 and the resumption of regular breathing 1.5, indicating the time it takes for the subject to recover normal breathing after holding their breath.

In some aspects, a trigger mechanism may be configured to initiate a disturbance of the ventilatory control system by instructing the user to hold their breath. This breath hold duration 1.3 can be a volitional action by the user, initiated upon instruction from the system. The length of the breath hold duration, T₁ 1.3 can vary depending on the user's comfort and ability, and can be adjusted as part of the breath training exercise.

In some cases, a processor may be further configured to analyze the physiological parameters to determine a recovery time duration and a number of recovery breaths following a breath-holding event. The recovery time duration Tr can be calculated as the time interval between the end of the breath hold duration 1.3 and the resumption of regular breathing 1.5. The number of recovery breaths can be determined by analyzing the breathing pattern during the resumption of regular breathing 1.5. In this first example, Tr is short indicative of a low LG. This information can be used to assess the stability of the user's ventilatory control system and to tailor the breath training exercise to the user's specific requirements.

In other aspects, the trigger mechanism may initiate a disturbance of the ventilatory control system by instructing the user to hold their breath for a specific duration. This breath hold duration 1.3 can be predetermined by the system based on the user's physiological parameters and the desired outcome of the breath training exercise. The user may be instructed to hold their breath for a longer duration as their ventilatory control system becomes more stable and their ability to hold their breath improves.

In yet other cases, the processor may be further configured to analyze the physiological parameters to determine a recovery time duration and a number of recovery breaths following a breath-holding event of a specific duration. The recovery time duration Tr and the number of recovery breaths can be used to estimate the loop gain of the user's ventilatory control system, which can provide beneficial information for diagnosing sleep disorders and tailoring treatment strategies.

Turning now to FIG. 1B, a graph is depicted representing a breathing pattern during a breath training exercise, over a period of time. The graph illustrates the transition from the end of a relaxed breathing sequence 1.7 to a breath hold duration T₂ 1.9, followed by the first recovery breath 1.11. The end of the relaxed breathing sequence 1.7 shows regular breathing patterns, which then cease during the breath hold duration 1.9, and resume with an increased amplitude at the first recovery breath 1.11, indicating the subject's response to the breath hold. The recovery time duration Tr and the number of recovery breaths can be used to estimate the loop gain of the user's ventilatory control system, which can provide beneficial information for diagnosing sleep disorders and tailoring treatment strategies. In this example, the recovery time, Tr, is slightly longer than that depicted in FIG. 1A, indicating somewhat greater breath control instability.

In some aspects, the end of the relaxed breathing sequence 1.7 may represent a state of calm and regular breathing prior to the initiation of a breath hold. This state may be achieved through various methods, such as deep breathing exercises or meditation techniques. The regularity and rhythm of the breathing pattern during this sequence may be indicative of the user's level of relaxation and control over their breathing.

In some cases, the breath hold duration 1.9 may be a volitional action by the user, initiated upon instruction from the system. The length of the breath hold duration 1.9 can vary depending on the user's comfort and ability and can be adjusted as part of the breath training exercise. The breath hold duration 1.9 may serve as a trigger to disturb the ventilatory control system, allowing for the assessment of the system's stability and the user's ability to control their breathing.

In other aspects, the first recovery breath 1.11 may represent the resumption of breathing following the breath hold. The amplitude of the first recovery breath 1.11 may be increased compared to the relaxed breathing sequence 1.7, indicating a response to the breath hold. The number of recovery breaths following the breath hold, including the first recovery breath 1.11, may be used to assess the stability of the user's ventilatory control system and their ability to recover normal breathing after a disturbance.

In yet other cases, variations to these patterns may be observed. For instance, the length of the relaxed breathing sequence 1.7, the breath hold duration 1.9, and the number of recovery breaths may vary depending on the user's physiological parameters and the desired outcome of the breath training exercise. Furthermore, the amplitude and frequency of the breathing patterns during the relaxed breathing sequence 1.7 and the recovery breaths may also vary, providing additional information about the user's ventilatory control system and their response to the breath hold.

Turning to FIG. 1C, a graph is depicted representing a breathing pattern during a breath training exercise, over a period of time. The graph illustrates the transition from the end of a relaxed breathing sequence 1.13 to the start of breath holding 1.15, followed by the breath holding duration T₃, and concluding with the start of an over breathing sequence 1.17. The relaxed breathing sequence 1.13 is characterized by regular, rhythmic waveforms, which abruptly cease at the start of breath holding 1.15, initiating the breath holding duration T₃. After the breath holding duration T₃, the pattern shifts to an over breathing sequence 1.17, 1.15 indicated by larger amplitude waveforms, signifying a change in breathing intensity.

In some aspects, the end of the relaxed breathing sequence 1.13 may represent a state of calm and regular breathing prior to the initiation of a breath hold. This state may be achieved through various methods, such as deep breathing exercises or meditation techniques. The regularity and rhythm of the breathing pattern during this sequence may be indicative of the user's level of relaxation and control over their breathing.

In some cases, the start of breath holding 1.15 may be a volitional action by the user, initiated upon instruction from the system. The length of the breath holding duration T₃ can vary depending on the user's comfort and ability and can be adjusted as part of the breath training exercise. The breath holding duration T₃ may serve as a trigger to disturb the ventilatory control system, allowing for the assessment of the system's stability and the user's ability to control their breathing.

In other aspects, the start of an over breathing sequence 1.17 may represent the resumption of breathing following the breath hold. The amplitude of the breathing during the over breathing sequence 1.17 may be increased compared to the relaxed breathing sequence 1.13, indicating a response to the breath hold. The number of breaths during the over breathing sequence 1.17 may be used to assess the stability of the user's ventilatory control system and their ability to recover normal breathing after a disturbance. The recovery time duration Tr and the number of recovery breaths can be used to estimate the loop gain of the user's ventilatory control system, which can provide beneficial information for diagnosing sleep disorders and tailoring treatment strategies. In this example, the recovery time, Tr, is longer than that depicted in FIGS. 1A and 1B.

In yet other cases, variations to these patterns may be observed. For instance, the length of the relaxed breathing sequence 1.13, the breath hold duration T₃, and the number of breaths during the over breathing sequence 1.17 may vary depending on the user's physiological parameters and the desired outcome of the breath training exercise. Furthermore, the amplitude and frequency of the breathing patterns during the relaxed breathing sequence 1.13 and the over breathing sequence 1.17 may also vary, providing additional information about the user's ventilatory control system and their response to the breath hold.

Referring now to FIG. 1D, a graph is depicted representing a breathing pattern during a breath-holding exercise, over a period of time. The graph illustrates the transition from the end of a relaxed breathing sequence 1.19 to the initiation of a breath hold, represented by the breath hold duration T₄, 1.21, and then the start of resumed breathing 1.23. The breath hold time span T₄ spans the duration of the breath hold, from the end of the relaxed breathing sequence 1.19, to the start of resumed breathing 1.23, indicating the subject's response to the breath-holding exercise.

In some aspects, the end of the relaxed breathing sequence 1.19 may represent a state of calm and regular breathing prior to the initiation of a breath hold. This state may be achieved through various methods, such as deep breathing exercises or meditation techniques. The regularity and rhythm of the breathing pattern during this sequence may be indicative of the user's level of relaxation and control over their breathing.

In some cases, the breath hold duration 1.21 may be a volitional action by the user, initiated upon instruction from the system. The length of the breath hold duration 1.21 can vary depending on the user's comfort and ability and can be adjusted as part of the breath training exercise. The breath hold duration 1.21 may serve as a trigger to disturb the ventilatory control system, allowing for the assessment of the system's stability and the user's ability to control their breathing.

In other aspects, the start of resumed breathing 1.23 may represent the resumption of breathing following the breath hold. The amplitude and frequency of the breathing during the resumed breathing sequence may vary, indicating a response to the breath hold. The number of breaths during the resumed breathing sequence may be used to assess the stability of the user's ventilatory control system and their ability to recover normal breathing after a disturbance. The recovery time duration Tr and the number of recovery breaths can be used to estimate the loop gain of the user's ventilatory control system, which can provide beneficial information for diagnosing sleep disorders and tailoring treatment strategies. In this example, the recovery time, Tr, is longer than that depicted in FIGS. 1A, 1B and IC.

In yet other cases, variations to these patterns may be observed. For instance, the length of the relaxed breathing sequence 1.19, the breath hold duration 1.21, and the number of breaths during the resumed breathing sequence may vary depending on the user's physiological parameters and the desired outcome of the breath training exercise. Furthermore, the amplitude and frequency of the breathing patterns during the relaxed breathing sequence 1.19 and the resumed breathing sequence may also vary, providing additional information about the user's ventilatory control system and their response to the breath hold.

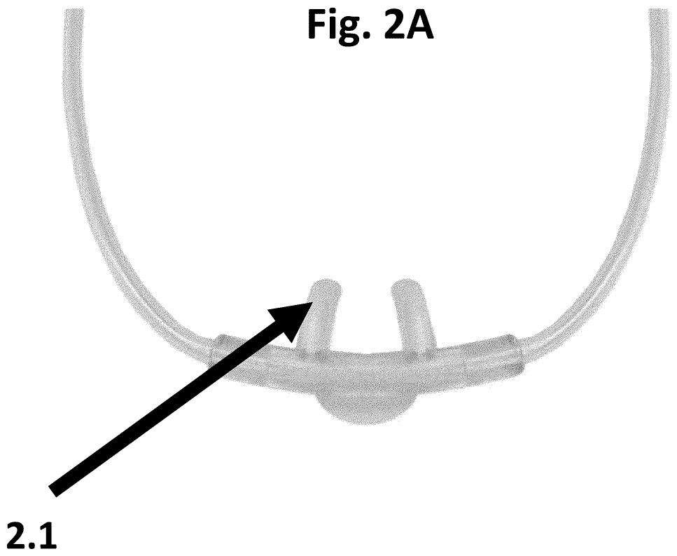

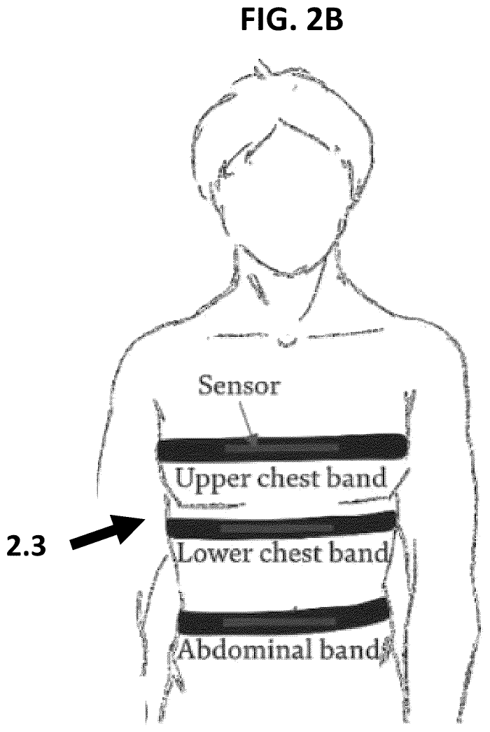

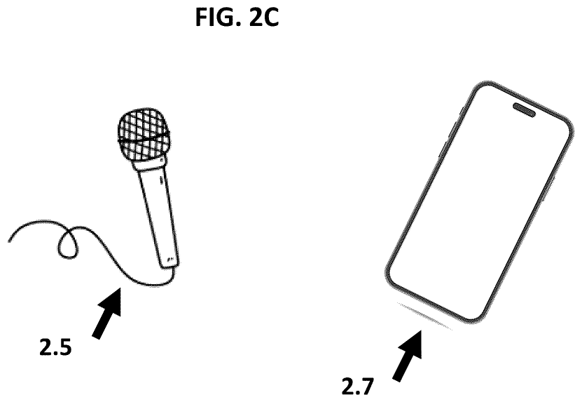

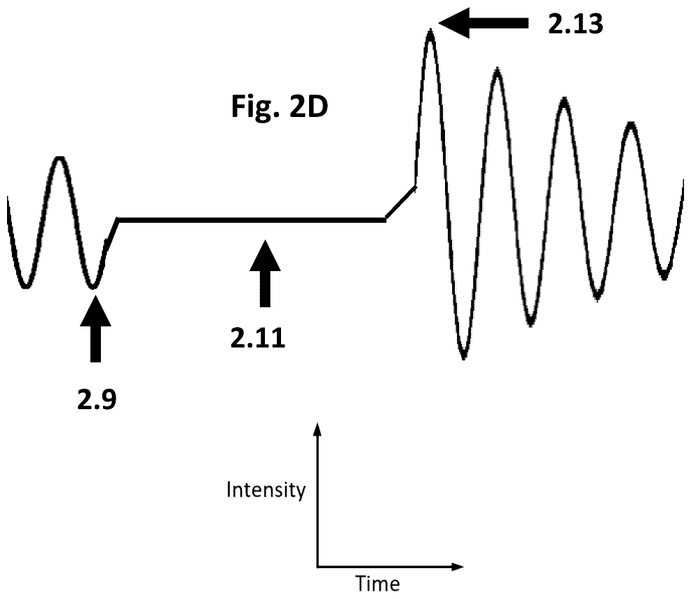

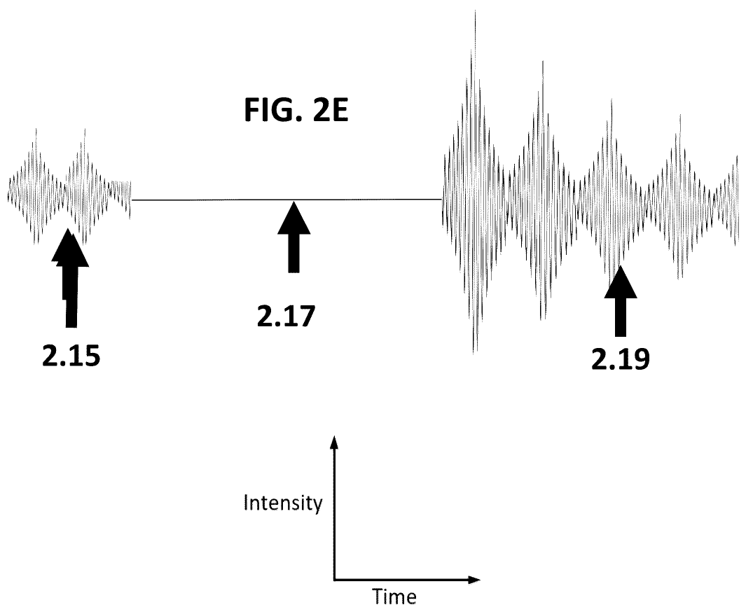

Turning to FIG. 2A, a front view of nasal cannula prongs 2.1 is depicted. In some aspects, the nasal cannula prongs 2.1 may be designed to fit into a user's nostrils. This configuration allows for the detection of breathing patterns, which can be monitored for various respiratory assessments as part of the described system. The nasal cannula prongs 2.1 may be connected to a device capable of recording and analyzing the user's breathing patterns, providing beneficial data for the assessment of the user's ventilatory control system.

In some cases, the nasal cannula prongs 2.1 may be made of a flexible material to ensure a comfortable fit for the user. The size and shape of the nasal cannula prongs 2.1 may also vary to accommodate different users. The nasal cannula prongs 2.1 may be designed to reduce or even minimize interference with the user's normal breathing, allowing for accurate detection of the user's natural breathing patterns.

In other aspects, the nasal cannula prongs 2.1 may be used in conjunction with other sensors or devices to provide a comprehensive assessment of the user's respiratory function. For instance, the nasal cannula prongs 2.1 may be used together with a heart rate monitor or a blood oxygen sensor to provide additional physiological data. This data can be used to enhance the accuracy of the loop gain and arousal index estimations, and to tailor the breath training exercises to the user's specific requirements.

Referring now to FIG. 2B, a human figure is depicted wearing a lower chest band 2.3. In some aspects, the lower chest band 2.3 may be positioned around the lower chest area of the human figure, indicating its placement for detecting breathing movements. The lower chest band 2.3 may be designed to expand and contract with the user's breathing, allowing for the detection and recording of the user's breathing patterns.

In some cases, the lower chest band 2.3 may be made of a flexible and elastic material to ensure a comfortable fit for the user. The size and shape of the lower chest band 2.3 may also vary to accommodate different users. The lower chest band 2.3 may be designed to reduce or even minimize interference with the user's normal activities, allowing for accurate detection of the user's natural breathing patterns even during physical activity or sleep.

In other aspects, the lower chest band 2.3 may be used in conjunction with other sensors or devices to provide a comprehensive assessment of the user's respiratory function. For instance, the lower chest band 2.3 may be used together with a heart rate monitor or a blood oxygen sensor to provide additional physiological data. This data can be used to enhance the accuracy of the loop gain and arousal index estimations, and to tailor the breath training exercises to the user's specific requirements.

In yet other cases, variations to these patterns may be observed. For instance, the lower chest band 2.3 may be positioned at different locations on the user's torso, such as the upper chest or abdomen, depending on the user's comfort and the desired outcome of the breath training exercise. Furthermore, the lower chest band 2.3 may be used in combination with other types of breathing sensors, such as nasal cannula prongs or microphones, to provide a more comprehensive assessment of the user's breathing patterns.

Turning to FIG. 2C, a schematic layout of a free-standing microphone 2.5 and a smartphone 2.7 is depicted. In some aspects, the free-standing microphone 2.5 may be positioned to capture audio independently. This configuration allows for the detection and recording of sounds generated by the movement of air entering and leaving the airways during breath training or sleep. The free-standing microphone 2.5 may be designed to filter out background noises and other artifacts, providing a clear and accurate recording of the user's breathing sounds.

In some cases, the smartphone 2.7 may be used in place of or in conjunction with the free-standing microphone 2.5 to detect and record the user's breathing sounds. The smartphone 2.7 may be equipped with a built-in microphone capable of capturing audio data. The smartphone 2.7 may also include a processor configured to analyze the recorded sounds and extract relevant information, such as the frequency and amplitude of the user's breathing patterns. This information can be used to assess the stability of the user's ventilatory control system and to tailor the breath training exercises to the user's specific requirements.

In other aspects, the smartphone 2.7 may be configured to detect and record additional physiological parameters, including but not limited to heart rate, heart rate variability, blood pressure, and blood oxygen concentration. This data can provide a comprehensive assessment of the user's physiological state, enhancing the accuracy of the loop gain and arousal index estimations, and providing beneficial insights for the diagnosis and treatment of sleep disorders.

In yet other cases, variations to these configurations may be observed. For instance, other types of portable smart devices, such as tablets or wearable devices, may be used in place of or in conjunction with the smartphone 2.7 to detect and record physiological parameters. Furthermore, other types of microphones, such as directional or omnidirectional microphones, may be used in place of or in conjunction with the free-standing microphone 2.5 to capture the user's breathing sounds. These variations provide flexibility and adaptability, allowing the system to be tailored to the specific requirements of different users and applications.

Referring now to FIG. 2D, a graph is depicted representing a waveform over a period of time starting with a period of relaxed breathing, 2.9, which leads to a period of relative quiet, typical of reduced or termination of breathing, 2.11, followed by a secondary characteristic peak 2.13. The relationship between these elements illustrates the progression and features of the waveform. In some aspects, the relative quiet period, 2.11, may indicate a notable feature in the waveform, such as a period of volitionary breath holding, or a period of restricted breathing as determined by the user's breathing sounds.

In some cases, the secondary characteristic peak 2.13 may represent an additional notable feature within the same waveform. This secondary peak may correspond to a different phase of the user's breathing cycle, such as the transition between a voluntary or involuntary breath hold and inhalation or exhalation. As one example, the time between 2.9 and 2.13 represents the length of a voluntary or involuntary reduction or cessation of breathing. The presence and characteristics of the secondary peak. 2.13 and subsequent peaks and troughs may provide additional insights into the user's breathing patterns, such as the time and rhythm of breathing recovery after a voluntary or involuntary reduction or cessation of breathing or the presence of any irregularities or disturbances.

In other aspects, the waveform analysis may be used to detect and record the user's breathing sounds during both awake and sleep states. The waveform start point 2.9, the characteristic quiet period, 2.11, and the secondary characteristic peak 2.13 may be used to analyze the frequency and amplitude of the user's breathing sounds, providing a comprehensive assessment of the user's ventilatory control system. This information can be used to estimate the loop gain of the user's ventilatory control system, and to tailor the breath training exercises to the user's specific requirements.

In yet other cases, variations to these patterns may be observed. For instance, the waveform may include additional peaks or troughs, indicating further features or phases of the user's breathing cycle. The waveform may also vary in shape, amplitude, or frequency, reflecting variations in the user's breathing patterns. These variations provide flexibility and adaptability, allowing the system to be tailored to the specific requirements of different users and applications.

Referring now to FIG. 2E, a diagram is depicted illustrating breathing sounds over a period time for the purpose of analyzing breathing patterns. The initial raw audio data may include sounds generated by the user's breathing, as well as other sounds present in the environment. In some aspects, the initial sound recording may be captured using a microphone, such as a free-standing microphone or a microphone integrated into a portable smart device, such as a smartphone.

The sound recording may be processed which may involve various techniques, such as noise reduction, signal enhancement, and frequency analysis, to isolate the user's breathing sounds from other sounds and to extract features of interest. In some cases, the processing stage may be performed by a processor integrated into the portable smart device, or by a separate processing unit.

In this example the result of this processing is the extracted breathing frequencies 2.15 and 2.19, which display the breathing patterns in a more refined form, emphasizing the frequencies and amplitudes pertinent to the user's breathing. These extracted breathing frequencies and amplitudes 2.15 and 2.19 are interspersed by a period of reduced sound, 2.17, and are representative of a voluntary or involuntary breath-hold in between periods of normal breathing. The diagram in FIG. 2E illustrates how detected sounds may be used to derive the breathing pattern illustrated in FIG. 2D. Such sound detection may provide beneficial information about the user's ventilatory control system, such as the regularity and rhythm of their breathing, the depth and rate of their breaths, and any irregularities or disturbances in their breathing patterns. In some aspects, the extracted breathing frequencies 2.19, and the quiet period 2.17 may be used to estimate the loop gain and arousal index of the user's ventilatory control system, and to tailor the breath training exercises to the user's specific requirements.

In yet other cases, variations to these patterns may be observed. For instance, the initial sound recording 2.15 may include other physiological sounds, such as heartbeats or body movements, in addition to the user's breathing sounds. The processing stage may employ different techniques or algorithms, depending on the characteristics of the initial sound recording and the desired outcome of the analysis. The extracted breathing frequencies, 2.15 and 2.19 may vary in shape, amplitude, or frequency, reflecting variations in the user's breathing patterns. These variations provide flexibility and adaptability, allowing the system to be tailored to the specific requirements of different users and applications.

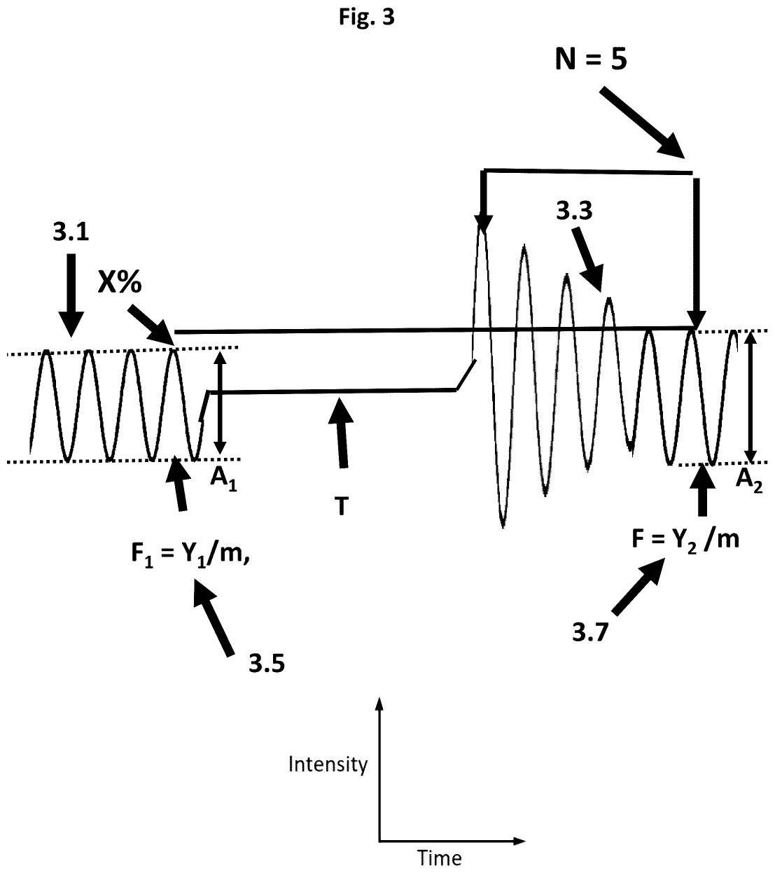

Referring now to FIG. 3, a graph is depicted illustrating the calculation of estimated Loop Gain (LG) in a breathing pattern, over a period of time. The pre-breathhold amplitude, A₁, 3.1 may represent the amplitude of the user's breathing before the initiation of a breath hold. This amplitude may be determined by analyzing the user's breathing patterns during a period of relaxed breathing prior to the breath hold. The pre-breathhold amplitude A₁ 3.1, may provide beneficial information about the user's normal breathing patterns, which can be used as a baseline for assessing changes in the user's breathing following a breath hold.

In some aspects, the pre-breathhold amplitude, A₁ 3.1 and the post-breathhold amplitude, A₂, 3.3 indicate a substantial change in breathing amplitude following a breath-holding event. The initial frequency, F₁, 3.5. is the number of breaths per minute calculated by detecting Y₁ breaths, (e.g., 5 breaths), and dividing by the time m over which these breaths occur. The breath amplitude prior to a breathhold, A₁, is the mean amplitude detected over several pulses, typically 5 breaths. F₁ and A₁ are stored as references to compare with the post breathhold breathing recovery pattern data. Similarly, the recovery frequency formula 3.7 is applied to determine the breathing frequency, F₂, during the recovery phase after the breath-hold where F₂ is the number of breaths per minute calculated by detecting Y₂ breaths, and dividing by the time m over which these breaths occur. The duration of the breath-hold is represented by T, and the number of recovery breaths, indicated by N, is used to assess the ventilatory control system's stability.

In some cases, the post-breathhold amplitude, A₂, 3.3 may represent the amplitude of the user's breathing following a breath hold. This amplitude may be determined by analyzing the user's breathing patterns during the recovery phase after the breath hold. The post-breathhold amplitude, A₂ 3.3 may provide beneficial information about the user's response to the breath hold, including the user's ability to recover normal breathing and the stability of the user's ventilatory control system. Several breaths, N may be required to return to frequency F₁ and A₁, and a value X %, (e.g. 10%), is added to amplitude A₁, to determine the value of A₂, at which sufficient recovery has occurred. The number of pulses from the end of the breathhold to the first pulse at which A₂ is detected is designated N, a key parameter for the determination of loop gain. The recovery value, N, may provide beneficial information about the user's response to the breath hold, including the user's ability to recover normal breathing and the stability of the user's ventilatory control system.

In some aspects, the duration of the breath hold, represented by T, and the number of recovery breaths, indicated by N, may be used to assess the stability of the user's ventilatory control system. These parameters may be used to estimate the loop gain and the arousal index of the user's ventilatory control system, providing beneficial information for diagnosing sleep disorders and tailoring treatment strategies. In some cases, the processor may be further configured to estimate the loop gain and the arousal index based on these physiological parameters and the response of the ventilatory control system to the disturbance.

The disclosed system is designed to identify specific patient phenotypes related to sleep disorders, particularly those associated with Sleep Disordered Breathing (SDB) and Obstructive Sleep Apnea (OSA). These phenotypes may include, but are not limited to, 1) poor musculature causing the airways to close, which is a mechanical impairment of the upper airway leading to increased collapsibility of the airway, 2) a central nervous system problem causing the body to stop breathing, which is characterized by a high loop gain contributing to unstable ventilatory control during sleep, and 3) a low arousal index, which is defined by a low respiratory arousal threshold contributing to unstable ventilatory control. The system is also capable of identifying patients who exhibit a combination of these phenotypes, providing a comprehensive assessment of the patient's condition and enabling the tailoring of treatment strategies to the specific requirements of the patient.

In one example, a software application installed on a smartphone is utilized to monitor and analyze a patient's breathing patterns. This application is designed to listen to the patient's breath during specific activities such as breath training exercises or while the patient is asleep. The smartphone's built-in microphone serves as a sensor, capturing the sounds generated by the patient's breathing. These sounds, which vary in frequency and amplitude, are then processed and analyzed by the application.

The software application is capable of distinguishing between different breathing patterns and classifying them into specific phenotypes. This classification is based on the characteristics of the patient's breathing, such as the rhythm, depth, and regularity of breaths, as well as the response to breath-holding exercises. By monitoring these parameters, the application can identify specific phenotypes associated with sleep disorders, such as poor musculature causing the airways to close, a central nervous system problem causing the body to stop breathing, or a low arousal index. This information can then be used to tailor treatment strategies to the specific requirements of the patient, improving the effectiveness of sleep disorder therapies.

The determination of these phenotypes can be achieved by monitoring one or more of a number of physiological signals. These signals may include, but are not limited to, heart rate, heart rate variability, blood pressure, blood oxygen concentration, and changes in breathing patterns (e.g. recorded sound). The analysis of these physiological signals can provide valuable insights into the patient's ventilatory control system, enabling the estimation of loop gain and arousal index, and the identification of specific phenotypes associated with sleep disorders. For example, a user with a loop gain above a predetermined threshold, as determined by analyzing the number of recovery breaths and breath hold duration during a voluntary breath-holding exercise, may be classified as having a high loop gain phenotype. This phenotype may be associated with central nervous system instabilities causing periodic breathing. Conversely, a user with an arousal index below a certain threshold, as determined by monitoring physiological responses to external triggers during sleep, may be classified as having a low arousal threshold phenotype. This phenotype may be indicative of sleep fragmentation and insomnia. In cases where a user exhibits a normal loop gain and a normal arousal index, but shows signs of airway obstruction during sleep, they may be classified as having a mechanical upper airway impairment phenotype. Users with a combination of low arousal threshold and high respiratory drive may be classified as having an unstable respiratory control phenotype. Those with normal loop gain and arousal threshold but exhibiting frequent arousals may be classified as having a non-respiratory arousal disorder phenotype. The system may also identify combined phenotypes, such as a user with both high loop gain and low arousal threshold, or a user with mechanical upper airway impairment and high loop gain, which may require tailored treatment approaches addressing multiple aspects of their sleep disorder.

In one example, the system can utilize sound signals as the sole source of physiological data. These sound signals can be captured using a microphone, such as a free-standing microphone or a microphone integrated into a portable smart device, such as a smartphone. The microphone serves as a sensor, capturing the sounds generated by the patient's breathing. These sounds, which vary in frequency and amplitude, are then processed and analyzed by the system. The system is capable of distinguishing between different breathing patterns based on the characteristics of the captured sound signals. By monitoring the frequency and amplitude of these sound signals, the system can identify specific phenotypes associated with sleep disorders. For instance, the rhythm, depth, and regularity of the patient's breathing sounds can provide valuable information about the patient's ventilatory control system, enabling the identification of specific phenotypes such as poor musculature causing the airways to close, a central nervous system problem causing the body to stop breathing, or a low arousal index.

The disclosed system effectively provides a novel non-invasive approach to diagnosing sleep disorders by determining the loop gain and arousal index of a patient's ventilatory control system. The loop gain is determined by instructing the patient to hold their breath during a breath training exercise. The patient's smartphone, equipped with a specialized software application, listens to the patient's breath to determine the duration of the breath hold and the recovery time. The duration of the breath hold and the number of recovery breaths are used to estimate the loop gain, a measure of the stability of the patient's ventilatory control system. A high loop gain may indicate a central nervous system problem causing the body to stop breathing, one of the phenotypes associated with sleep disorders.

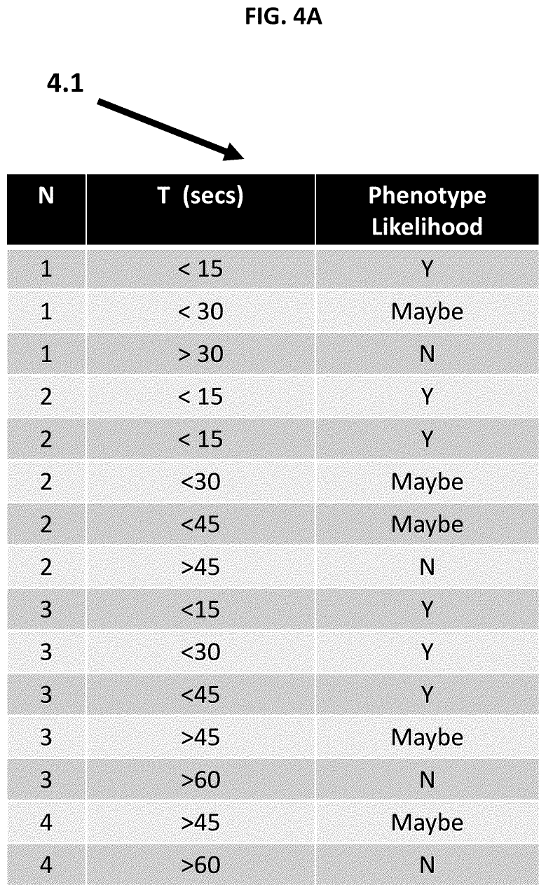

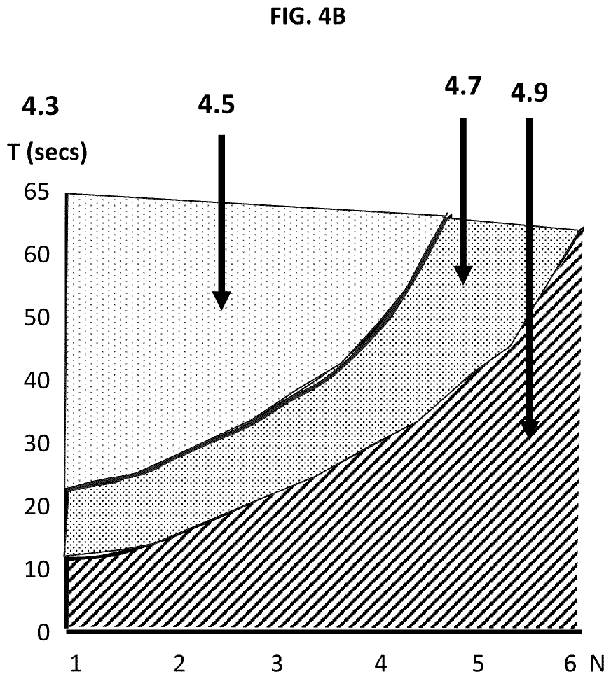

Referring now to FIG. 4A, a table is depicted that provides a guideline for determining the likelihood of a patient's phenotype based on breath holding time (T in seconds) and the number of recovery breaths (N). The table header 4.1 is positioned at the top of the table, indicating the categories of information presented in the columns below. The table lists various combinations of N and T values, with corresponding assessments of ‘Y’ for Yes, ‘Maybe’, or ‘N’ for No, to indicate the likelihood of a particular phenotype related to ventilatory control system stability.

In some aspects, the table may be used to interpret patient data in the context of the described system. The relationship between the N and T values and the phenotype likelihood is laid out in the table, providing a reference for interpreting patient data. For instance, a patient with a lower number of recovery breaths (N) and a longer breath holding time (T) may be more likely to have a phenotype characterized by a stable ventilatory control system, as indicated by a ‘Yes’ assessment in the table. Conversely, a patient with a higher number of recovery breaths (N) and a shorter breath holding time (T) may be more likely to have a phenotype characterized by an unstable ventilatory control system, as indicated by a ‘No’ assessment in the table.

In other cases, the table may be used to guide the selection of appropriate treatment strategies for patients with sleep disorders. For instance, a patient with a phenotype characterized by an unstable ventilatory control system may benefit from breath training exercises designed to enhance their control over their breathing, while a patient with a phenotype characterized by a stable ventilatory control system but exhibiting reduced airway patency may require different treatment strategies, such as the use of a positive airway pressure device or surgical interventions.

In yet other aspects, variations to these patterns may be observed. For instance, the table may include additional categories of information, such as the patient's age, gender, or other physiological parameters, to provide a more comprehensive assessment of the patient's phenotype. Furthermore, the table may include additional assessments, such as ‘Likely’, ‘Unlikely’, or ‘Uncertain’, to provide a more nuanced interpretation of the patient's data. These variations provide flexibility and adaptability, allowing the system to be tailored to the specific requirements of different users and applications.

Turning now to FIG. 4B, a graph is depicted illustrating the relationship between recovery breaths and the time taken to recover after a breath-holding exercise, as part of a method to estimate Loop Gain (LG) in patients. The graph shows a curve with data points marked at various stages of recovery breaths. These data points represent different recovery times after a breath-holding exercise, indicating the patient's ventilatory control system response.

In some aspects, the data point at lower recovery breaths 4.3 represents a stage where the patient has taken a lower number of recovery breaths N after a breath-holding exercise. This may suggest a lower LG value, indicating a more stable ventilatory control system. In other cases, the data point at mid recovery breaths 4.5 represents a stage where the patient has taken a moderate number of recovery breaths after a breath-holding exercise. This may suggest a moderate LG value, indicating a moderately stable ventilatory control system.

In some aspects, the data point at higher recovery breaths 4.7 represents a stage where the patient has taken a higher number of recovery breaths after a breath-holding exercise. This may suggest a higher LG value, indicating a less stable ventilatory control system. In other cases, the data point at the highest recovery breaths 4.9 represents a stage where the patient has taken the maximum number of recovery breaths after a breath-holding exercise. This may suggest an even higher LG value, which may exceed 1, indicating a less stable ventilatory control system.

In yet other cases, variations to these patterns may be observed. For instance, the number of recovery breaths and the time taken to recover after a breath-holding exercise may vary depending on the patient's physiological parameters and the desired outcome of the breath training exercise. Furthermore, the LG values associated with different stages of recovery breaths may also vary, providing additional information about the patient's ventilatory control system and their response to the breath hold.

The arousal index, on the other hand, is determined by applying external triggers such as sound, vibration, or light during the patient's sleep. The smartphone monitors the patient's response to these triggers to assess how easily the patient is aroused from sleep. Physiological parameters such as heart rate or breathing patterns are monitored to detect arousal from sleep. A low arousal index may indicate a low respiratory arousal threshold, another phenotype associated with sleep disorders. Once the phenotype diagnosis is performed based on the determined loop gain and arousal index, treatment can be tailored to the patient's specific requirements, improving the effectiveness of sleep disorder therapies.

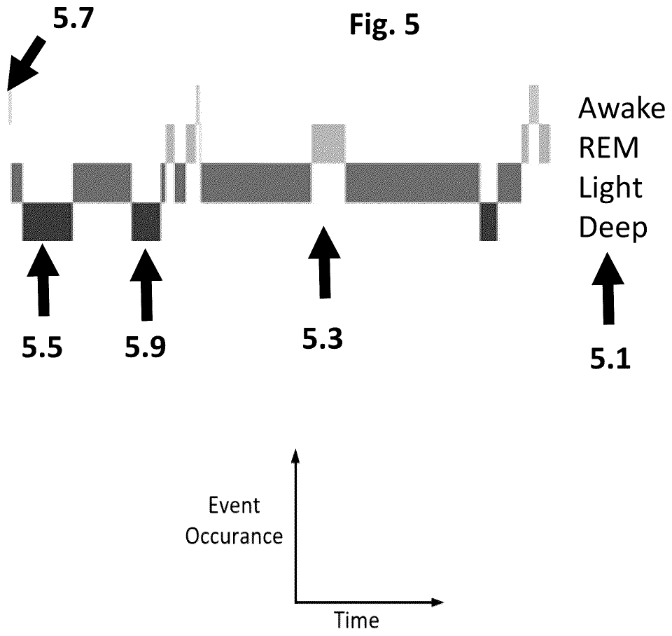

Referring now to FIG. 5, a graph is depicted illustrating four stages of sleep, 5.1, over a period of time. The graph shows, after a short period required to fall asleep referred to as latency, 5.7, a first deep sleep stage 5.5, a later period of deep sleep 5.9, and a later Rapid Eye Movement (REM) sleep stage 5.3. The arrangement of these stages on the graph reflects the typical progression of sleep stages throughout the night.

In some aspects, the first deep sleep stage 5.5 may represent the first phase of deep sleep that a user enters after falling asleep. This stage is characterized by slow brain waves known as delta waves, and it is during this stage that the body performs many restorative functions, such as tissue repair and hormone release. The initial deep sleep stage 5.5 may be followed by the early deep sleep stage 5.9, which may represent a subsequent phase of deep sleep. The user may cycle between these deep sleep stages and other stages of sleep throughout the night.

In some cases, the early deep sleep stages, 5.5 and 5.9, may occur earlier in the sleep cycle, while the later REM sleep stage 5.3 is positioned towards the latter part of the sleep cycle. REM sleep is characterized by rapid eye movements, increased brain activity, and vivid dreams. It is during the later REM sleep stage 5.3 that the user may experience the majority of their dreaming.

In other aspects, the progression of sleep stages as depicted in FIG. 5 may vary depending on a variety of factors, including the user's age, health status, and sleep environment. For instance, the latency, 5.7, the duration and frequency of the initial deep sleep stage 5.5, the early deep sleep stage 5.9, and the later REM sleep stage 5.3 may vary from night to night and from user to user. Furthermore, the progression of sleep stages may be influenced by factors such as the user's sleep schedule, exposure to light, and consumption of caffeine or alcohol.

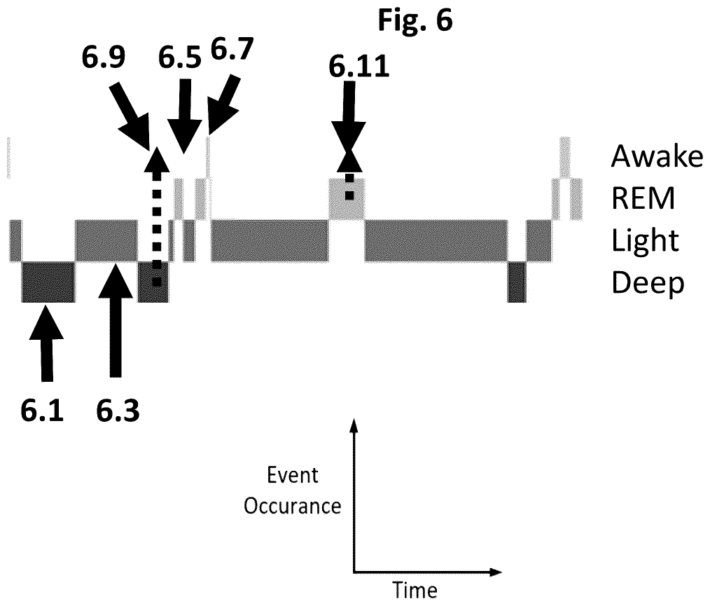

Referring now to FIG. 6, a diagram is depicted illustrating the different sleep stages and the transition to an arousal state, over a period of time as part of a method for monitoring sleep and breathing patterns. The diagram shows a sequence of deep sleep mode 6.1 transitioning to light sleep mode 6.3, followed by REM sleep mode 6.5, and finally to awake mode 6.7. An arousal state indicator 6.9 is used to represent the point at which the patient is aroused from deep sleep. An arousal state indicator 6.11 is used to represent the point at which the patient is aroused from REM sleep. The arrows indicate the direction of transition between the different sleep modes and the arousal state, with the dotted arrows suggesting the application of an external trigger that leads to the arousal state. The introduction of sleep stages in the disclosed system/method is a strategic approach to accurately determine the arousal index of a patient's ventilatory control system. It is partially based on the understanding that applying triggers for arousal index during light or awake states may not yield meaningful results because these states may not provide the requisite conditions for a reliable assessment of the patient's response to external triggers. Therefore, the disclosed system/method is designed to monitor sleep stages during sleep, for example, focusing on deep sleep and REM sleep stages. These stages are periods of sleep where the body is in its deepest rest and where dreams occur, respectively. By applying triggers during these stages, the disclosed system/method may provoke a response from the ventilatory control system that is indicative of the patient's arousal threshold. This approach applies triggers under conditions that are conducive to a reliable and accurate assessment of the patient's ventilatory control system stability.

In some aspects, the deep sleep mode 6.1 may represent a phase of sleep characterized by slow brain waves and decreased physiological activity. During this mode, the user may be less responsive to external stimuli, and disturbances to the ventilatory control system may have a greater impact on the user's sleep quality. The transition from deep sleep mode 6.1 to light sleep mode 6.3 may occur naturally as part of the user's sleep cycle, or it may be induced by an external trigger as part of the described system.

In some cases, the light sleep mode 6.3 may represent a phase of sleep characterized by increased physiological activity and responsiveness to external stimuli compared to deep sleep mode 6.1. The transition from REM sleep to awake sleep mode 6.11 may occur naturally as part of the user's sleep cycle, or it may be induced by a trigger as part of the described system.

In yet other cases, the arousal state indicator 6.9 may represent the point at which the user is aroused from sleep. This may occur as a result of a trigger applied during deep sleep mode 6.9 or REM sleep mode 6.11, or it may occur naturally as part of the user's sleep cycle. The arousal state indicators 6.9 and 6.11 may be used to monitor the user's response to the trigger and to assess the stability of the user's ventilatory control system.

In some aspects, the transitions between the different sleep modes and the arousal state may vary depending on a variety of factors, including the user's age, health status, and sleep environment. For instance, the duration and frequency of the deep sleep mode 6.1, the light sleep mode 6.3, and the REM sleep mode 6.5 may vary from night to night and from user to user. Furthermore, the timing and intensity of the triggers applied during deep sleep mode 6.9 or REM sleep mode 6.11 may also vary, providing flexibility and adaptability in the monitoring and assessment of sleep and breathing patterns.

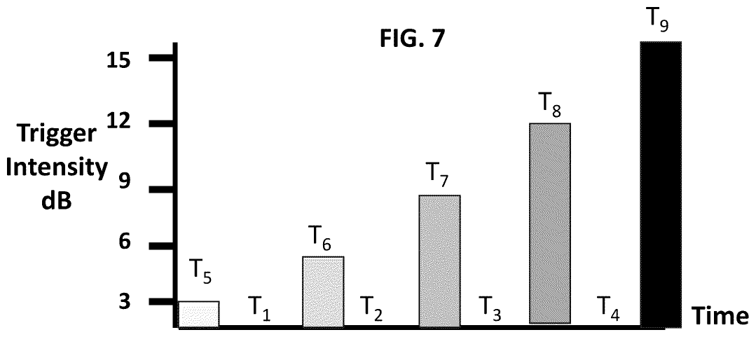

Referring now to FIG. 7, a graph is depicted illustrating a sequence of trigger applications over time. The graph shows a series of triggers with increasing intensity, starting with a baseline trigger intensity T5, a second stage trigger intensity T6, a mid-level trigger intensity T7, a penultimate trigger intensity T8, and culminating in a peak trigger intensity T9. Each trigger is separated by a specific duration, with the second intensity trigger T6 occurring after a time duration T1, and subsequent triggers T6, T7, T8, and T9 occurring at regular intervals or nearly regular intervals due to slight variations, each marked by a corresponding time label T2, T3, T4, and so on. The triggers are represented as bars on the graph, with their height corresponding to the trigger intensity in decibels (dB), illustrating a stepwise increase in intensity designed to assess the arousal threshold during sleep. Human responses to external stimuli are known to follow a decibel scale, 3 dB corresponding to a doubling of the stimulus intensity.

In some aspects, the trigger mechanism may be configured to initiate a disturbance of the ventilatory control system by applying an external trigger. This external trigger may at least one of sound, light, and motion. The intensity of the external trigger may vary, starting with an initial low intensity trigger T5 and increasing in a stepwise manner to a peak trigger intensity T9. This stepwise increase in intensity is designed to progressively challenge the ventilatory control system, allowing for a comprehensive assessment of the system's stability and the user's arousal threshold.

In the context of the disclosed system for diagnosing and treating sleep disorders, a “motion” trigger can be utilized as an external stimulus to initiate a disturbance of the ventilatory control system. This motion trigger can be generated by an external device that is capable of producing a physical sensation, such as vibration or touch, which can create a response by the patient.

The motion trigger operates by delivering a physical stimulus to the patient during specific stages of sleep, particularly during deep sleep or REM sleep. The stimulus is designed to provoke a response from the patient's ventilatory control system, thereby causing a disturbance in the patient's autonomous breathing pattern. The response of the ventilatory control system to this disturbance is then monitored and analyzed to determine the patient's arousal index and/or loop gain, which are indicative of the stability of the ventilatory control system.

Examples of external devices that can generate a motion trigger include, but are not limited to, wearable devices such as smartwatches or fitness trackers, bed-mounted devices, or even specialized medical devices. These devices can be equipped with actuators or motors that are capable of producing a physical sensation, such as vibration or touch. For instance, a smartwatch equipped with a haptic feedback mechanism can be programmed to deliver a series of vibrations to the patient's wrist at specific intervals during the sleep cycle. The intensity and frequency of these vibrations can be adjusted to increase progressively, thereby challenging the ventilatory control system and allowing for a comprehensive assessment of its stability. Similarly, a bed-mounted device can be designed to generate a motion trigger by producing a physical movement in the bed, such as a gentle shake or a rhythmic pulsation. This movement can be perceived by the patient, thereby initiating a disturbance of the ventilatory control system.

In other words, the use of a motion trigger as an external stimulus provides a non-invasive and effective method for diagnosing sleep disorders and tailoring treatment strategies. By monitoring the patient's response to these triggers, valuable insights can be gained into the stability of the patient's ventilatory control system, thereby enabling the identification of specific phenotypes associated with sleep disorders and the development of personalized treatment plans.

In some cases, the timing of the trigger applications may be determined based on the user's physiological parameters and the desired outcome of the sleep study. For instance, the low intensity trigger T6 may be applied after a recovery time duration T1, allowing the user's ventilatory control system to return to a baseline state before the application of the trigger. Subsequent triggers T7, T8, and T9 may be applied at regular or nearly regular intervals, each marked by a corresponding time label T2, T3, T4, and so on. This regular timing or nearly regular timing due to slight variations of the trigger applications may facilitate the monitoring of the user's response to the triggers and the assessment of the ventilatory control system's stability.

In other aspects, the intensity of the triggers may be represented as bars on the graph, with their height corresponding to the trigger intensity in decibels (dB). This visual representation of the trigger intensity may provide a clear and intuitive depiction of the stepwise increase in intensity, facilitating the interpretation of the user's response to the triggers and the assessment of the ventilatory control system's stability.

In yet other cases, variations to these patterns may be observed. For instance, the intensity and timing of the triggers may vary depending on the user's physiological parameters and the desired outcome of the sleep study. Furthermore, the type of external trigger applied may vary, including but not limited to sound, light, and motion. These variations provide flexibility and adaptability, allowing the system to be tailored to the specific requirements of different users and applications.

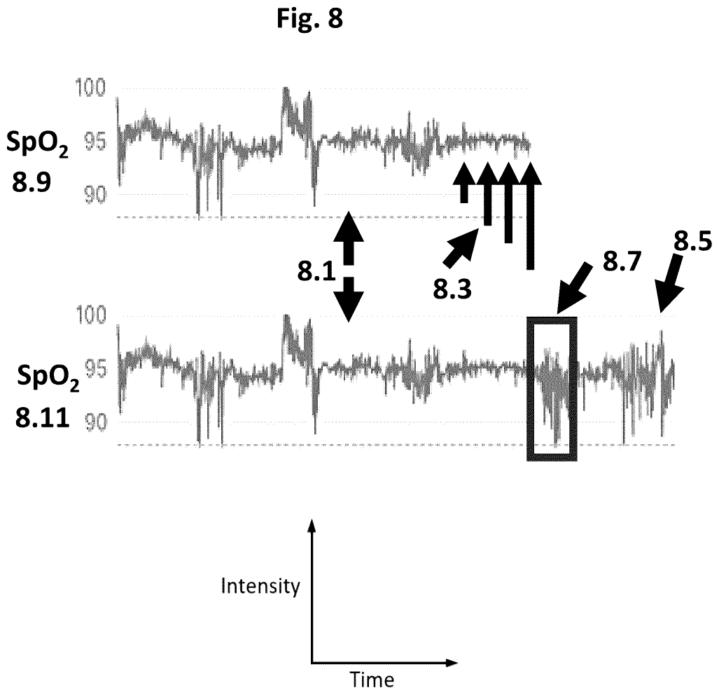

Referring now to FIG. 8, a graph is depicted illustrating the response of a patient's blood oxygen saturation (SpO₂) during sleep to various external triggers, over a period of time. The upper trace, 8.9, shows SpO₂ during sleep until a sequence of external triggers are applied. The lower trace, 8.11, shows SpO₂ during sleep until a sequence of external triggers are applied and the response of SpO₂ thereafter. Baseline SpO₂ stability 8.1 is shown during a period of relatively stable blood oxygen levels, indicating calm deep sleep. In some aspects, the baseline SpO₂ stability 8.1 may be determined using a pulse oximetry device, which measures the concentration of oxygen in the patient's blood. This device may be used to identify periods of deep sleep, during which the patient's SpO₂ levels are typically stable and high.

Trigger intensity series 8.3 represents a sequence of external stimuli applied with increasing intensity, designed to provoke a response. In some cases, these external triggers may be generated by a trigger mechanism integrated into the system. The intensity of the triggers may vary, starting with a low intensity trigger and increasing in a stepwise manner. The timing and intensity of the triggers may be determined based on the patient's physiological parameters and the desired outcome of the sleep study.

SpO₂ variability response 8.7 follows the application of the triggers, showing fluctuations in blood oxygen levels as a reaction to the stimuli. In some aspects, the SpO₂ variability response 8.7 may be used to assess the stability of the patient's ventilatory control system and their response to the triggers. The SpO₂ variability response 8.5 may be monitored and recorded by a pulse oximetry device, providing beneficial data for the analysis of the patient's ventilatory control system.

A subsequent notable arousal event 8.5 is indicated by a substantial decrease in SpO₂, suggesting an arousal from deep sleep. In some cases, the notable arousal event 8.5 may be used to identify instances of arousal from deep sleep, which may be indicative of a low arousal threshold not caused by an external trigger The notable arousal event 8.5 may be monitored and recorded by a pulse oximetry device, providing beneficial data for the estimation of the patient's arousal index.

In yet other cases, the processor may be further configured to track the loop gain and the arousal index over time. This tracking may be based on the physiological parameters detected and recorded by the portable smart device, including the SpO₂ levels and the response of the ventilatory control system to the triggers. The tracking of the loop gain and the arousal index over time may provide beneficial insights into the progression of the patient's sleep disorder and the effectiveness of the treatment strategies.

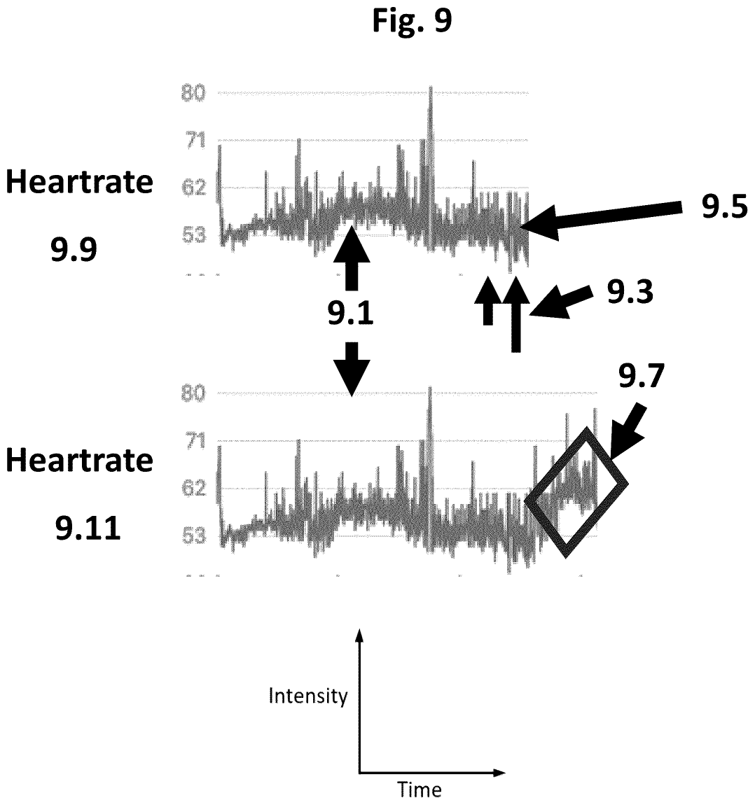

Referring now to FIG. 9, a graph is depicted illustrating heart rate monitoring during sleep, over a period of time. The upper trace, 9.9, shows heartrate during sleep until a sequence of external triggers are applied at 9.3. The lower trace, 9.11, shows heartrate during sleep until a sequence of external triggers are applied and the response of heartrate thereafter. The baseline heart rate 9.1 is shown at the beginning of the graph, establishing the normal heart rate level before any external triggers are applied. In some aspects, the baseline heart rate 9.1 may be determined using a heart rate sensor, which measures the user's heart rate at regular intervals. This sensor may be integrated into a wearable device, such as a wristband, a ring or a chest strap, or it may be a standalone device placed in close proximity to the user.

A slow reduction in heart rate 9.5 suggests the user has entered a deep sleep phase. In some cases, the deep sleep indicator 9.5 may be determined based on the user's heart rate and other physiological parameters, such as body temperature or movement. The deep sleep indicator 9.5 may provide beneficial information about the user's sleep cycle, which can be used to tailor the timing and intensity of the external triggers.

At the external trigger application point 9.3, at period of deep sleep. a sequence of triggers are introduced. In some aspects, this intervention may be an external trigger applied by the system, such as a sound, light, or vibration. The timing and intensity of the external trigger may be determined based on the user's physiological parameters and the desired outcome of the sleep study. The external trigger application point 9.3 may serve as a disturbance to the ventilatory control system, allowing for the assessment of the system's stability and the user's arousal threshold.

Following the application of the external trigger, the arousal from deep sleep indicator 9.7 shows a noticeable increase in heart rate, indicating the user's response to the external trigger and arousal from deep sleep. In some cases, the arousal from deep sleep indicator 9.7 may be determined using a heart rate sensor, which measures the user's heart rate at regular intervals. The arousal from deep sleep indicator 9.7 may provide beneficial information about the user's response to the external trigger, including the user's ability to recover normal heart rate and the stability of the user's ventilatory control system.

In yet other cases, variations to these patterns may be observed. For instance, the baseline heart rate 9.1, the deep sleep indicator 9.5, the external trigger application point 9.3, and the arousal from deep sleep indicator 9.7 may vary depending on the user's physiological parameters and the desired outcome of the sleep study. Furthermore, the type of external trigger applied at the external trigger application point 9.3 may vary, including but not limited to sound, light, and vibration. These variations provide flexibility and adaptability, allowing the system to be tailored to the specific requirements of different users and applications.

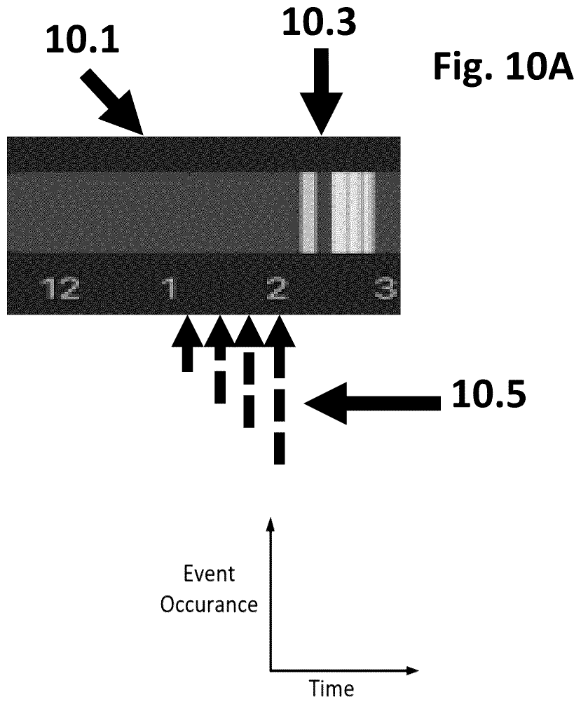

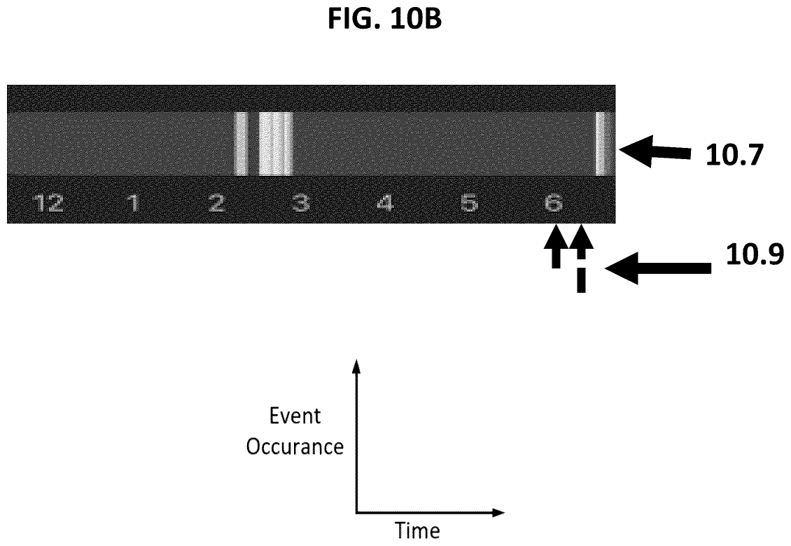

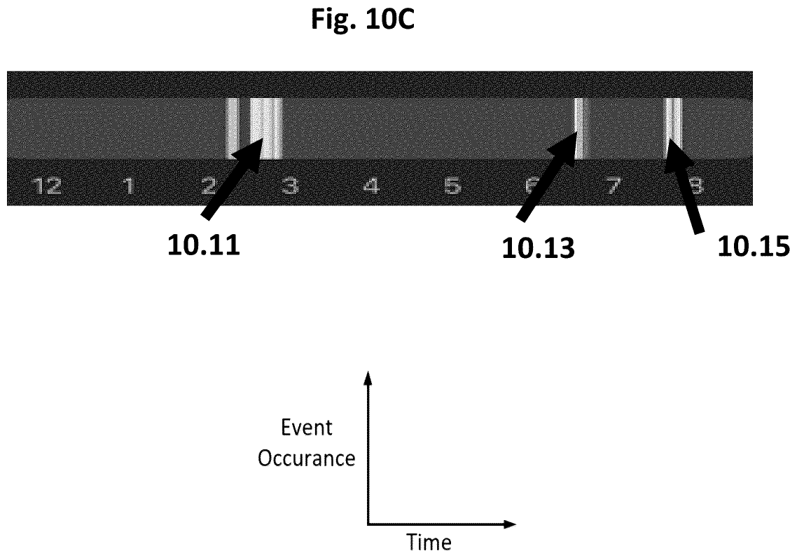

Referring now to FIG. 10A, a graph is depicted illustrating a breathing pattern and the application of intensity triggers, over a period of time during sleep. Periods of irregular breathing are indicated by light bands on the chart. The stable breathing pattern section 10.1 shows a consistent and regular pattern of breathing. This regular pattern may be indicative of a calm and relaxed state of the user, achieved through various methods such as deep breathing exercises or meditation techniques. The regularity and rhythm of the breathing pattern during this section may provide beneficial information about the user's level of relaxation and control over their breathing.

In some aspects, the irregular breathing response section 10.3 indicates a change in the breathing pattern, which becomes irregular in response to the applied triggers. This irregular pattern may be indicative of a disturbance to the ventilatory control system, caused by the application of external triggers. The irregularity and variability of the breathing pattern during this section may provide beneficial information about the user's response to the triggers and the stability of their ventilatory control system.

In some cases, the intensity trigger indicators 10.5 represent the points at which increasing levels of intensity are applied, leading to the observed irregular breathing response section 10.3. These triggers may be generated by a trigger mechanism integrated into the system, and their intensity may vary, starting with a low intensity trigger and increasing in a stepwise manner. The timing and intensity of the triggers may be determined based on the user's physiological parameters and the desired outcome of the sleep study.

In other aspects, the relationship between the stable breathing pattern section 10.1, the irregular breathing response section 10.3, and the intensity trigger indicators 10.5 demonstrates how the breathing pattern is affected by the applied triggers. This relationship may be used to estimate the arousal threshold (AT) during sleep, providing beneficial information for diagnosing sleep disorders and tailoring treatment strategies.

In yet other cases, variations to these patterns may be observed. For instance, the stable breathing pattern section 10.1, the irregular breathing response section 10.3, and the intensity trigger indicators 10.5 may vary depending on the user's physiological parameters and the desired outcome of the sleep study. Furthermore, the type of external trigger applied at the intensity trigger indicators 10.5 may vary, including but not limited to sound, light, and vibration. In some aspects, a microphone may be used to determine when a trigger is applied and a subsequent estimate of AT. These variations provide flexibility and adaptability, allowing the system to be tailored to the specific requirements of different users and applications.

Turning now to FIG. 10B, a diagram is depicted related to the monitoring of breathing patterns, over a period of time during sleep. Periods of irregular breathing are indicated by light bands on the chart. The diagram shows the relationship between various elements, including a breathing instability trigger point 10.9, a REM sleep measurement point 10.7, and a sequence timing axis. In some aspects, the breathing instability trigger point 10.7 may be associated with an event or condition that could disrupt normal breathing. This disruption may be caused by a variety of factors, such as a change in the user's physiological state, an external stimulus, or a malfunction in the ventilatory control system.

In some cases, the REM sleep measurement point 10.7 may suggest a specific time or condition during REM sleep when measurements are taken. These measurements may include various physiological parameters, such as heart rate, blood oxygen concentration, and changes in breathing patterns. The REM sleep measurement point 10.7 may provide beneficial information about the user's physiological state during REM sleep, which can be used to assess the stability of the user's ventilatory control system and to tailor the breath training exercises to the user's specific requirements.

In other aspects, the sequence timing axis may denote the beginning of a sequence or the identification of a component within the system. This may be used to track the progression of the user's sleep cycle, the application of external triggers, and the user's response to these triggers. The sequence timing axis may provide a reference point for the analysis of the user's breathing patterns and the assessment of the user's ventilatory control system.

In yet other cases, a microphone may be used to determine when a trigger is applied and a subsequent estimate of AT. This microphone may be integrated into a portable smart device, such as a smartphone, or it may be a standalone device placed in close proximity to the user. The microphone may be configured to detect and record the sounds generated by the user's breathing, providing a non-invasive and convenient method for monitoring the user's breathing patterns during sleep. The recorded sounds may be analyzed to determine the timing and intensity of the triggers, as well as the user's response to these triggers. This information can be used to estimate the arousal threshold (AT) during sleep, providing beneficial information for diagnosing sleep disorders and tailoring treatment strategies.

Turning now to FIG. 10C, a timeline representation of a sequence of events during sleep is depicted. The timeline starts with an initial hourly timeline marker 12, followed by a sequence of timeline markers at regular intervals: second timeline marker 1, third timeline marker 2, fourth timeline marker 3, fifth timeline marker 4, sixth timeline marker 5, seventh timeline marker 6, and eighth timeline marker 7. Arousal event indicators 10.11, 10.13 and 10.15 after positioned after the timeline markers 2, and 7, signifying arousal events during the sleep study. The arrangement of the timeline markers and the arousal event indicators illustrates the progression of time and the occurrence of arousal events within that timeline.

In some aspects, the timeline markers may represent specific points in time during the sleep study, such as the start and end of different sleep stages, the application of external triggers, or the occurrence of specific events, such as arousals or disturbances in the user's breathing patterns. The timeline markers may be used to track the progression of the sleep study and to provide a visual representation of the sequence of events. The timeline markers may be generated automatically by the system based on the user's physiological parameters and the timing of the external triggers.

In some cases, the arousal event indicators 10.11, 10.13 and 10.15 may represent specific events during the sleep study, such as arousals from sleep or a disturbance in the user's breathing patterns. The arousal event indicators 10.11, 10.13 and 10.15 may be determined based on the user's physiological parameters, such as changes in heart rate, blood oxygen concentration, or breathing patterns. The arousal event indicators 10.11, 10.13 and 10.15 may provide beneficial information about the user's response to the external triggers and the stability of their ventilatory control system.

In other aspects, a microphone may be used to determine when a trigger is applied and a subsequent estimate of AT. This microphone may be integrated into a portable smart device, such as a smartphone, or it may be a standalone device placed in close proximity to the user. The microphone may be configured to detect and record the sounds generated by the user's breathing, providing a non-invasive and convenient method for monitoring the user's breathing patterns during sleep. The recorded sounds may be analyzed to determine the timing and intensity of the triggers, as well as the user's response to these triggers. This information can be used to estimate the arousal threshold (AT) during sleep, providing beneficial information for diagnosing sleep disorders and tailoring treatment strategies.

In yet other cases, variations to these patterns may be observed. For instance, the timeline markers may be positioned at different intervals, depending on the user's physiological parameters and the desired outcome of the sleep study. Furthermore, the arousal event indicators 10.11, 10.13 and 10.15 may vary in position, depending on the timing and intensity of the triggers and the user's response to these triggers. In some aspects, a different type of sensor, such as a heart rate monitor or a blood oxygen sensor, may be used in place of or in conjunction with the microphone to detect and record physiological parameters. These variations provide flexibility and adaptability, allowing the system to be tailored to the specific requirements of different users and applications.

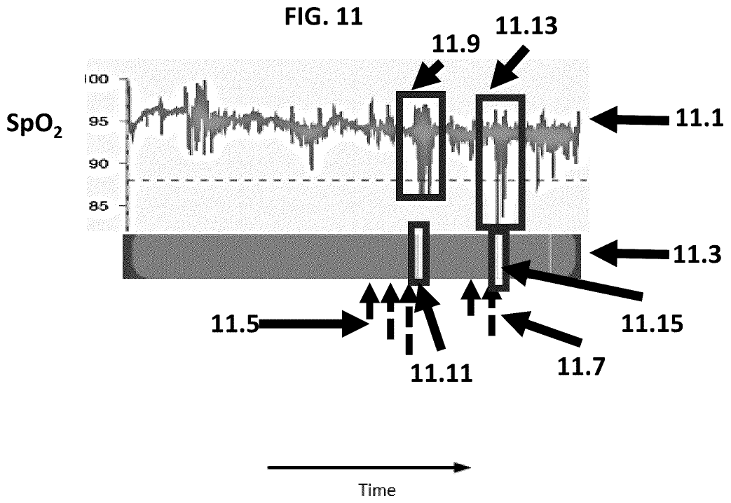

Turning now to FIG. 11, a graph is depicted illustrating the response of physiological parameters to a series of intensity triggers, over a period of time during sleep. The pulse oximeter trace 11.1 shows fluctuations in blood oxygen saturation levels, while the audio airflow trace 11.3 represents breathing sounds captured during the sleep study. The first intensity trigger 11.5, and second intensity trigger 11.7, are applied at different points in time, each followed by evidence of arousal, indicated by the first trigger arousal evidence 11.9, and second trigger arousal evidence 11.13. The relationship between the intensity triggers and the corresponding arousal evidence demonstrates the system's ability to detect and record the patient's response to external stimuli during sleep.

In some aspects, a pulse oximetry device may be used to monitor the patient's blood oxygen saturation levels during sleep, as represented by the pulse oximeter trace 11.1. This device may be configured to measure the concentration of oxygen in the patient's blood at short time intervals, providing real-time data on the patient's physiological state. The fluctuations in blood oxygen saturation levels as shown in the pulse oximeter trace 11.1 may provide beneficial information about the patient's ventilatory control system and their response to the external triggers.

In some cases, a microphone may be used to capture the patient's breathing sounds during sleep, as represented by the audio airflow trace 11.3. It is noted that airflow trace 11.3, may not directly indicate the airflow or the volume of air being inhaled or exhaled by the patient. Instead, it may represent the variations or instabilities in the patient's breathing patterns as detected and recorded by the system. The system, through the use of a portable smart device such as a smartphone, captures the sounds generated by the movement of air entering and leaving the patient's airways during both awake and sleep states. These sounds are then processed and analyzed to extract relevant information, such as the frequency and amplitude of the patient's breathing. In a stable or regular breathing pattern, the patient may have a consistent frequency of breaths, for example, 10 breaths per minute. However, during periods of instability, such as during an arousal event from deep sleep or during a breath-holding exercise, the frequency and amplitude of the patient's breathing may vary from this baseline. The airflow trace thus represents these variations or shifts from the baseline breathing pattern. For instance, during an arousal event, the patient's breathing frequency may increase to 20 breaths per minute, double the baseline rate (e.g., double 10 breaths per minute). Similarly, the amplitude of the patient's breathing, which represents the volume of air inhaled or exhaled during each breath, may also vary during these events. The microphone may be integrated into a portable smart device, such as a smartphone, or it may be a standalone device placed in close proximity to the patient. The microphone may be configured to detect and record the sounds generated by the movement of air entering and leaving the patient's airways, providing a non-invasive method for monitoring the patient's breathing patterns during sleep.

In other aspects, a series of external triggers may be applied during sleep to provoke a response from the patient's ventilatory control system. These triggers, represented by the first intensity triggers 11.5, and second intensity triggers 11.7, may be generated by a trigger mechanism integrated into the system. The intensity of the triggers may vary, starting with a low intensity trigger and increasing in a stepwise manner. The timing and intensity of the triggers may be determined based on the patient's physiological parameters and the desired outcome of the sleep study.

In yet other cases, the system may be configured to detect and record evidence of arousal following the application of the external triggers. This evidence of arousal, represented by the first trigger arousal evidence 11.9, second trigger arousal evidence 11.13, third trigger arousal evidence 11.11 and fourth trigger arousal evidence, 11.15, may be used to assess the stability of the patient's ventilatory control system and their arousal threshold. The timing and intensity of the arousal evidence may be determined based on the patient's physiological parameters and the response of the ventilatory control system to the triggers.

In some aspects, variations to these patterns may be observed. For instance, the pulse oximeter trace 11.1 and the audio airflow trace 11.3 may vary depending on the patient's physiological parameters and the desired outcome of the sleep study. Furthermore, the timing and intensity of the triggers and the arousal evidence may also vary, providing flexibility and adaptability in the monitoring and assessment of sleep and breathing patterns. These variations provide flexibility and adaptability, allowing the system to be tailored to the specific requirements of different users and applications.

In some aspects detecting arousal responses to triggers using two or more independent sensing means provides improved measurement of arousal thresholds. FIG. 11, at events 11.11 and 11.15, illustrates strong correlation between responses to triggers 11.5 and 11.7 by sensing both SpO₂ and breathing airflow. Cross correlation calculations of two or more sources of response related data may improve the accuracy of arousal threshold detection.

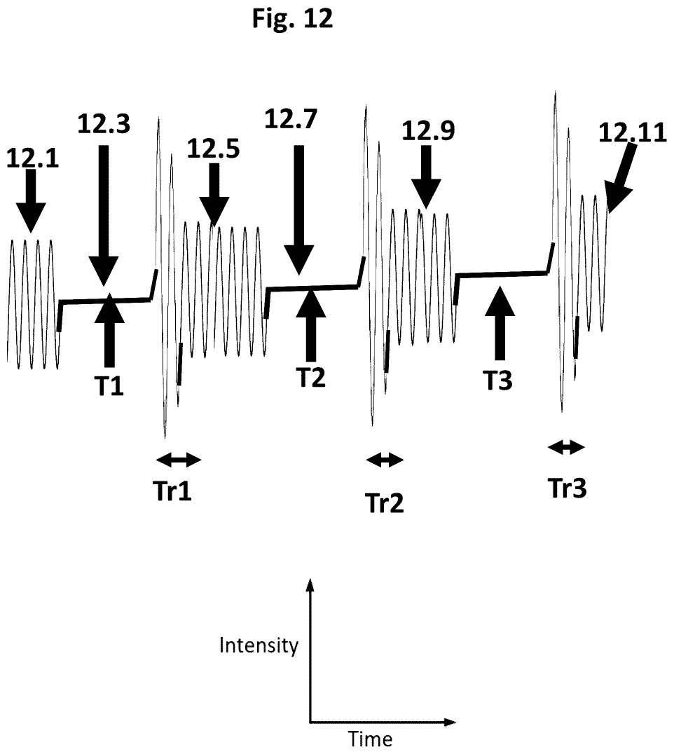

Turning now to FIG. 12, a graph is depicted illustrating a breathing exercise pattern over a period of time designed to alleviate sleep disruption due to breathing control instabilities. The graph shows the timing and amplitudes of a sequence of relaxed breathing followed by volitional breath holding. The first relaxed breathing sequence 12.1 transitions into the first volitional breath holding 12.3, followed by the second relaxed breathing sequence 12.5, which leads into the second volitional breath holding 12.7. The pattern continues with the third relaxed breathing sequence 12.9 and concludes with the fourth relaxed breathing sequence 12.11. The breath holding time duration T1 spans the interval between the start of the volitional breath hold and the first inhalation of the next relaxed breathing sequence, 12.5. Similarly, breath hold times, T2 and T3 are detected at subsequent breath-holds that occur after a period of relaxed breathing. Tr1, Tr2, Tr3 indicate the times taken for the amplitude of the breathing to return to within a defined percentage (e.g., 10%) of the relaxed breathing prior to the volitional breath-hold. Typically, a complete breath training exercise session encompasses up to six or more such sequences with interspersed relaxed breathing periods of one minute or more. The graph demonstrates the relationship between these breathing sequences and volitional breath holding, indicating a method for monitoring and improving breathing control.

In some aspects, the first relaxed breathing sequence 12.1 may represent a state of calm and regular breathing prior to the initiation of a breath hold. This state may be achieved through various methods, such as deep breathing exercises or meditation techniques. The regularity and rhythm of the breathing pattern during this sequence may be indicative of the user's level of relaxation and control over their breathing.

In some cases, the first volitional breath holding 12.3 may be a volitional action by the user, initiated upon instruction from the system. The length of the breath hold may vary depending on the user's comfort and ability and can be adjusted as part of the breath training exercise. The breath hold may serve as a trigger to disturb the ventilatory control system, allowing for the assessment of the system's stability and the user's ability to control their breathing.

In other aspects, the second relaxed breathing sequence 12.5 may represent a state of calm and regular breathing following the first volitional breath hold 12.3. The regularity and rhythm of the breathing pattern during this sequence may be indicative of the user's level of relaxation and control over their breathing. The recovery time duration Tr1, which spans the interval between the end of the first volitional breath holding 12.3 and reaching a defined percentage of breathing amplitude relative to the pre-breath-hold may be used to assess the stability of the user's ventilatory control system and their ability to recover normal breathing after a disturbance.

In yet other cases, a microphone may be used to monitor the breathing patterns during breath training. This microphone may be integrated into a portable smart device, such as a smartphone, or it may be a standalone device placed in close proximity to the user. The microphone may be configured to detect and record the sounds generated by the user's breathing, providing a non-invasive and convenient method for monitoring the user's breathing patterns during sleep. The recorded sounds may be analyzed to determine the timing and intensity of the triggers, as well as the user's response to these triggers. This information can be used to estimate the arousal threshold (AT) during sleep, providing beneficial information for diagnosing sleep disorders and tailoring treatment strategies.

As described above, the system may determine the LG of a patient's ventilatory control system by instructing the patient to hold their breath during a breath training exercise. This breath-holding event serves as a trigger to disturb the ventilatory control system. The patient's smartphone, equipped with a specialized software application, listens to the patient's breath to determine the duration of the breath hold and the recovery time. The duration of the breath hold and the number of recovery breaths are used to estimate the loop gain, a measure of the stability of the patient's ventilatory control system. A high loop gain may indicate a central nervous system problem causing the body to stop breathing, one of the phenotypes associated with sleep disorders.

The arousal index of the ventilatory control system is determined by applying external triggers such as sound, vibration, or light during the patient's sleep. The smartphone monitors the patient's response to these triggers to assess how easily the patient is aroused from sleep. Physiological parameters such as SpO₂, heart rate or breathing patterns are monitored to detect arousal from sleep. A low arousal index may indicate a low respiratory arousal threshold, another phenotype associated with sleep disorders.

The determination of LG and arousal threshold is beneficial in diagnosing specific phenotypes associated with sleep disorders. For instance, a high loop gain may indicate a central nervous system problem causing the body to stop breathing, while a low arousal index may indicate a low respiratory arousal threshold. These phenotypes are associated with sleep disorders such as SDB, OSA and insomnia.

Once the phenotype diagnosis is performed based on the determined LG and arousal index, treatment can be tailored to the patient's specific requirements, improving the effectiveness of sleep disorder therapies. For example, patients with a high LG phenotype may benefit from breath training exercises designed to enhance their control over their breathing, while patients with a low arousal index phenotype may require different treatment strategies, such as the use of one or more of a positive airway pressure device, mandibular advancement splint surgical interventions, or cognitive behavioral therapy. It is noted that in one example, CBT may be utilized for treatment for insomnia indicated by low arousal threshold.

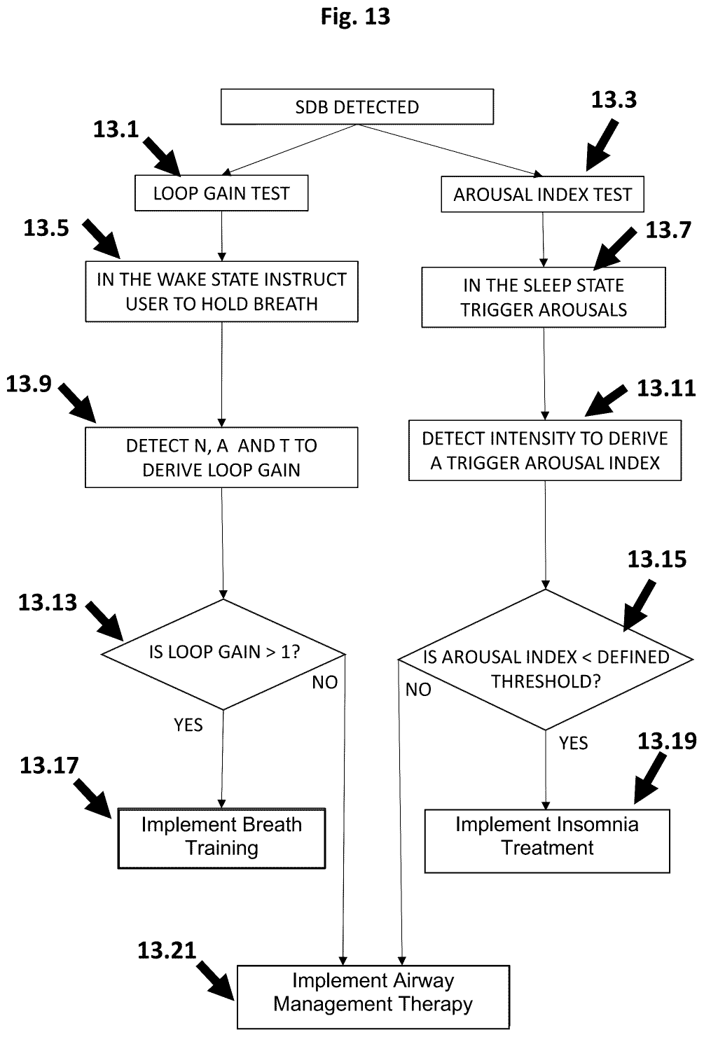

Turning now to FIG. 13, a flow chart of the system with key decision points is shown. It is first determined that a person is exhibiting alleviated disturbed breathing events during sleep. In order to determine which of several therapeutic interventions are indicated, the cause of the breathing disturbances is required. Three distinct phenotype categories, namely two arising from neuro-physical triggers, a high loop gain, and low arousal index and one arising from upper airway full or partial obstruction are considered. To determine which of these phenotypes are involved, the system detects whether high loop gain, 13.1 or low arousal index 13.3 are involved, and, by elimination, whether upper airway physical obstruction is the predominant cause.

One or both of high loop gain and/or low arousal index are determined by the methods described herein. In other words, the flowchart may proceed with steps 13.1, 13.5, 13.9, 13.13, 13.17, 13.21 and/or steps 13.3, 13.7, 13.11, 13.15, 13.19 13.21.

High loop gain is determined in the wake state using volitional breath hold triggers in step 13.5, detecting parameters to derive loop gain in step 13.9, and comparing loop gain to a threshold in step 13.13. If the loop gain is greater than the threshold, therapies such as breath training should be implemented in step 13.17. If loop gain is less than the threshold, breath training is less likely to be appropriate and other therapies such as airway management therapy in step 13.21 should be implemented.

On the other side of the flowchart, low arousal threshold is determined in the sleep state using externally applied triggers in step 13.7, detecting parameters to derive trigger arousal index in step 13.11, and comparing the trigger arousal index to a threshold in step 13.15. If the arousal index is less than the threshold, therapies best suited for insomnia should be implemented at step 13.19. If arousal index is greater than threshold, therapies targeted at improving upper airway patency should be implemented in step 13.21.

In other words, once the phenotype is determined, the system may recommend and implement a tailored treatment approach based on the specific characteristics of the identified phenotype or combination of phenotypes. For users with a high loop gain phenotype, the treatment may focus on breath training exercises designed to stabilize the respiratory control system, such as guided breathing patterns or CO2 rebreathing techniques. Those with a low arousal threshold phenotype may receive interventions aimed at improving sleep continuity, such as cognitive behavioral therapy for insomnia or the use of sedative medications. Users identified with mechanical upper airway impairment may be recommended treatments like positive airway pressure therapy, mandibular advancement devices, or in some cases, surgical interventions to improve airway patency. For combined phenotypes, the system may prioritize treatments based on the severity of each component or implement a multi-modal approach. For instance, a user with both high loop gain and mechanical impairment may receive a combination of breath training exercises and positive airway pressure therapy. The system may also continuously monitor the user's response to treatment through ongoing physiological measurements and periodic reassessments of loop gain and arousal threshold, allowing for dynamic adjustments to the treatment plan as needed. This personalized and adaptive approach aims to optimize treatment efficacy by addressing the specific underlying mechanisms of each user's sleep disorder.

While the foregoing is directed to example embodiments described herein, other and further example embodiments may be devised without departing from the basic scope thereof. Aspects of the present disclosure may be implemented in hardware or software or a combination of hardware and software. One example embodiment described herein may be implemented as a program product for use with a computer system. The program(s) of the program product defines functions of the example embodiments (including the methods described herein) and may be contained on a variety of computer-readable storage media. Illustrative computer-readable storage media include, but are not limited to: (i) non-writable storage media (e.g., read-only memory (ROM) devices within a computer, such as CD-ROM disks readably by a CD-ROM drive, flash memory, ROM chips, or any type of solid-state non-volatile memory) on which information is permanently stored; and (ii) writable storage media (e.g., floppy disks within a diskette drive or hard-disk drive or any type of solid-state random-access memory) on which alterable information is stored. Such computer-readable storage media, when carrying computer-readable instructions that direct the functions of the disclosed example embodiments, are example embodiments of the present disclosure.

It will be appreciated by those skilled in the art that the preceding examples are exemplary and not limiting. It is intended that all permutations, enhancements, equivalents, and improvements thereto are apparent to those skilled in the art upon a reading of the specification and a study of the drawings are included within the true spirit and scope of the present disclosure. It is therefore intended that the following appended claims include all such modifications, permutations, and equivalents as fall within the true spirit and scope of these teachings.

1. A system for diagnosing and treating sleep disorders, comprising:
a portable smart device configured to detect and record physiological parameters of a user during awake and sleep states;
a processor configured to analyze the physiological parameters to determine at least one of a loop gain or an arousal index of a ventilatory control system of the user; and
a trigger mechanism configured to initiate a disturbance of the ventilatory control system during awake and sleep states and monitor a response of the ventilatory control system to such disturbance to determine a phenotype of the user.

2. The system of claim 1, wherein the portable smart device is configured to detect and record physiological parameters including at least one of heart rate, heart rate variability, blood pressure, blood oxygen concentration, and changes in breathing patterns.

3. The system of claim 1, wherein the portable smart device is a smartphone.

4. The system of claim 1, wherein the trigger mechanism is configured to initiate the disturbance of the ventilatory control system by instructing the user to hold their breath.

5. The system of claim 1, wherein the trigger mechanism is configured to initiate the disturbance of the ventilatory control system by applying an external trigger including at least one of sound, light, and motion.

6. The system of claim 2, wherein the processor is further configured to analyze the physiological parameters to determine a recovery time duration and a number of recovery breaths following a breath-holding event.

7. The system of claim 1, wherein the processor is further configured to estimate the at least one of the loop gain or the arousal index based on the physiological parameters and the response of the ventilatory control system to the disturbance.

8. The system of claim 1, wherein the processor is further configured to track the at least one of the loop gain or the arousal index over time.

9. The system of claim 1, wherein the processor is further configured to provide feedback to the user based on the determined loop gain and arousal index.

10. The system of claim 1, further comprising a user interface configured to display the determined at least one of the loop gain or the arousal index to the user.

11. A method for diagnosing and treating sleep disorders, comprising steps of:
detecting and recording physiological parameters of a user during awake and sleep states using a portable smart device;
analyzing the physiological parameters to determine at least one of a loop gain or an arousal index of a ventilatory control system of the user using a processor;
initiating a disturbance of the ventilatory control system during awake and sleep states using a trigger mechanism; and
monitoring a response of the ventilatory control system to the disturbance to determine a phenotype of the user.

12. The method of claim 11, wherein the portable smart device is a smartphone.

13. The method of claim 11, wherein the physiological parameters including at least one of heart rate, heart rate variability, blood pressure, blood oxygen concentration, and changes in breathing patterns.

14. The method of claim 11, wherein the trigger mechanism initiates the disturbance of the ventilatory control system by instructing the user to hold their breath.

15. The method of claim 11, wherein the trigger mechanism initiates the disturbance of the ventilatory control system by applying an external trigger including at least one of sound, light, and motion.

16. The method of claim 13, wherein the processor is further configured to analyze the physiological parameters to determine a recovery time duration and a number of recovery breaths following a breath-holding event.

17. The method of claim 11, wherein the processor is further configured to estimate the at least one of the loop gain or the arousal index based on the physiological parameters and the response of the ventilatory control system to the disturbance.

18. The method of claim 11, wherein the processor is further configured to track the at least one of the loop gain or the arousal index over time.

19. The method of claim 11, wherein the processor is further configured to provide feedback to the user based on the determined at least one of the loop gain or the arousal index.

20. The method of claim 11, further comprising a user interface configured to display the determined at least one of the loop gain or the arousal index to the user.
